(19) United States
(12) Patent Application Publication
(10) Pub. No.: US 2022/0273568 A1
(43) Pub. Date: Sep. 1, 2022

(54) MULTIVESICULAR LIPOSOME FORMULATIONS OF DEXMEDETOMIDINE

(71) Applicant: Pacira Pharmaceuticals, Inc., San Diego, CA (US)

(72) Inventors: Soroush M. Ardekani, San Diego, CA (US); Patrick GHL. Boen, San Diego, CA (US); Louie D. Garcia, San Diego, CA (US); Paige N. Davis, San Diego, CA (US); Hassan G. Hussein, San Diego, CA (US); Kathleen D. A. Los, San Diego, CA (US)

(21) Appl. No.: 17/625,705

(22) PCT Filed: Jul. 9, 2020
(86) PCT No.: PCT/US2020/041379

§ 371 Date: Jan. 7, 2022

Related U.S. Application Data
(60) Provisional application No. 62/873,417, filed on Jul. 12, 2019.

(51) Int. Cl. A61K 9/127; A61K 31/4174; A61K 9/00; A61P 25/22; A61P 29/00; A61K 33/42

(52) U.S. Cl. CPC A61K 9/1275; A61K 31/4174; A61K 9/0019; A61P 25/22; A61P 29/00; A61K 33/42

(57) ABSTRACT

Some embodiments of the present application are related to multivesicular liposome formulations comprising dexmedetomidine (DXM) for the purpose of minimizing the side effects of immediate release formulation of dexmedetomidine while lengthening the duration of the effect with clinically meaniningful efficacy. Processes of making and administering DXM encapsulated multivesicular liposome formulations (DXM-MVLs) and their uses as medicaments are also provided.

### INCORPORATION BY REFERENCE TO ANY PRIORITY APPLICATIONS

The present application claims the benefit of priority to U.S. Provisional Appl. No. 62/873,417, filed Jul. 12, 2019, which is incorporated by reference in its entirety.

### BACKGROUND

### Field

The present disclosure relates to multivesicular liposome (MVL) formulations of dexmedetomidine (DXM), uses thereof and processes of making the same.

### Description of the Related Art

Dexmedetomidine is a very versatile drug that has been shown to be efficacious for a wide variety of indications, including: sedative, anxiolytic, analgesic, and sympatholytic. See Naaz, Journal of Clinical and Diagnostic Research. 2014; 8(10):GE01-GE04. It may be used in pre-, intra- and post-operative, in addition to palliative care settings. See Hilliard, Palliative Medicine. 2015; 29(3):278-281; and Su, The Lancet. 2016; 388(10054):1893-1902. Dexmedetomidine may be administered orally, systemically and locally, however, oral bioavailability is low, and the drug is typically cleared rapidly (t_{1/2}=2h), therefore a commonly used route of administration is intravenous (IV) continuous infusion. IV infusions are generally restricted to inpatient use, and are associated with various complications (blockage, infection, infiltration, phlebitis, inflammation, thrombosis, bruising, hematoma, etc.). Subcutaneous administration of a long-acting dexmedetomidine formulation eliminates infusion-associated complications, and provides added flexibility with regard to the setting for administration. In addition, subcutaneous administration has been shown to reduce the variability of plasma dexmedetomidine levels (which is often associated with onset of side effects), and to increase the duration of dexmedetomidine in the bloodstream, as compared to intravenous administration. See Saari, European Journal of Clinical Pharmacology. 2018; 74:1047-1054. Steady systemic dexmedetomidine levels are associated with reduced hemodynamic effects (e.g. tachycardia, hypertension) and increased rousability in sedated patients.

Accordingly, there is a need for a stable, sustained release formulation of DXM as an alternative to the commonly used IV route, allowing the patient to avoid the complications of IV infusion. Also, there is a need for a formulation with a minimum amount of, or essentially free of, unencapsulated dexmedetomidine to provide analgesia for pain management, yet preventing side effects associated with dexmedetomidine, such as sedation beyond the arousable state. The long-acting formulation would allow patients to receive a single injection. The multivesicular liposome formulations described herein address these needs and provide other advantages as well.

### BRIEF DESCRIPTION OF THE FIGURES

FIG. 1 is a line chart illustrating the dose normalized dexmedetomidine plasma levels obtained in rats as a function of time, following administration of several dexmedetomidine encapsulated multivesicular liposome (DXM-MVL) formulations as compared to dexmedetomidine in a saline solution.

FIG. 2A is a line chart illustrating the dose normalized dexmedetomidine plasma levels obtained in rats as a function of time, following administration of several DXM-MVL formulations varying internal pH.

FIG. 2B is a line chart illustrating the amount of drug released, in rats, during the first 24 h (% total AUC) as a function of MVL particle internal pH for various DXM-MVL formulations with varied internal pH, as seen in FIG. 2A.

FIG. 3A is a line chart illustrating the dose normalized dexmedetomidine plasma levels in dogs as a function of time, following SC administration of several DXM-MVL formulations with varying particle internal pH.

FIG. 3B is a line chart illustrating the amount of drug released, in dogs, during the first 24 h (% total AUC) as a function of MVL particle internal pH for various DXM-MVL formulations, as seen in FIG. 3A.

FIG. 4A is a line chart illustrating the cumulative AUC as a function of time of several DXM-MVL formulations with various lipid concentrations.

FIG. 4B is an enlarged portion of the line chart shown in FIG. 4A for the first 72 hours.

FIG. 4C is a line chart illustrating the dose normalized dexmedetomidine plasma level obtained in dogs as a function for time, following administration of several DXM-MVL formulations, as seen in FIG. 4A.

FIG. 5 is a line chart illustrating the internal pH of several DXM-MVL formulations as a function of initial (first aqueous solution) phosphoric acid concentrations when two different concentrations of dipalmitoylphosphatidylglycerol (DPPG) were used in the DXM-MVL formulations.

### SUMMARY

Embodiments of the present application relate to formulations comprising dexmedetomidine encapsulated multivesicular liposomes, processes of making the same, and uses thereof. Multivesicular liposome formulation of dexmedetomidine intended to provide sustained release of dexmedetomidine over the span of 2 to 14 days or 3 to 7 days, prolonging the therapeutic effect of the dexmedetomidine and leading to clinically meaningful efficacy while minimizing the undesirable side effects of immediate release formulations of dexmedetomidine. Processes of making multivesicular liposomes containing DXM and their use as medicaments are also provided.

Some embodiments of the present application relate to multivesicular liposome formulations encapsulating dexmedetomidine (DXM-MVL), the formulations include dexmedetomidine encapsulated in a first aqueous component of the multivesicular liposomes, a lipid component comprising at least one amphipathic lipid and at least one neutral lipid, and one or more pH modifying agents. In some embodiments, the formulation also comprises unencapsulated dexmedetomidine, also known as free dexmedetomidine.

Some embodiments of the present application relate to pharmaceutical compositions comprising the DXM-MVL formulations described herein. In some embodiments, the pharmaceutical composition is in the form of a suspension with dexmedetomidine encapsulated MVL particles suspended in a saline solution. In some embodiments, the saline solution is a buffered solution. In some embodiments, the pharmaceutical composition is for administration in a single injection. In some embodiments, a single injection of the pharmaceutical composition containing DXM-MVL may provide sustained release of dexmedetomidine for at least two days, for example, two to 14 days, or three to seven days.

Some embodiments of the present application relate to a method of treating or ameliorating anxiety or pain, comprising administering a pharmaceutical composition containing multivesicular liposomes encapsulating dexmedetomidine as described herein to a subject in need thereof. Some other embodiments of the present application relate to a method of inducing arousable sedation in a subject, comprising administering a pharmaceutical composition containing multivesicular liposomes encapsulating dexmedetomidine as described herein. In some embodiments, the treatments are for the purpose of providing palliative care to patients, in particular for pain and anxiety management. In some embodiments, the treatments described herein may also prevent or reduce the hemodynamic complications of pain and anxiety, such as hypotension or hypertension.

Some embodiments of the present application relate to a process for preparing dexmedetomidine encapsulated multivesicular liposomes, the process comprising: mixing a first aqueous component with a lipid component comprising at least one organic solvent, at least one amphipathic lipid, and at least one neutral lipid to form a first water-in-oil emulsion, wherein at least one of the first aqueous components and/or the lipid component comprises dexmedetomidine; combining the first water-in-oil emulsion with a second aqueous component to form a second emulsion; and substantially removing the organic solvent from the second emulsion to form multivesicular liposomes. In some embodiments, the process further includes diluting the second emulsion in a third aqueous solution prior to substantially removing the organic solvent. In some embodiments, the process further includes isolating the multivesicular liposome particles and suspending them in a liquid suspending medium (e.g., a buffered saline solution) to form a suspension of multivesicular liposomes.

Further embodiments of the present application relate to a pharmaceutical composition comprising dexmedetomidine encapsulated multivesicular liposomes prepared by the process described herein.

### DETAILED DESCRIPTION

Dexmedetomidine is an anxiety reducing, sedative, and pain medication. The currently approved dexmedetomidine product is sold under the tradename Precedex®, and is most often used in the intensive care setting for light to moderate sedation. Dexmedetomidine has analgesic properties in addition to its role as a hypnotic, but is opioid sparing. The present application provides pharmaceutical compositions comprising multivesicular liposomes encapsulating dexmedetomidine (DXM-MVLs) encapsulated in the internal aqueous chambers of the MVLs. A single dose of DXM-MVL composition may be administered once every 3 to 7 days for the treatment of pain and anxiety. This eliminates the need for continuous IV infusions, which are generally restricted to inpatient use, and which can be associated with various complications (blockage, infection, infiltration, phlebitis, inflammation, thrombosis, bruising, hematoma, etc.).

The present embodiments also provide the processes of preparing the DXM-MVLs and the methods of using the DXM-MVL formulations for treating, ameliorating or preventing pain, anxiety, or the hemodynamic complications of pain and anxiety (such as hypertension), comprising administering a DXM-MVLs pharmaceutical composition, as described herein, to a subject in need thereof, are disclosed herein. Some embodiments provide methods for inducing arousable sedation in a patient comprising administering a DXM-MVL pharmaceutical composition, as described herein, to said subject in need thereof are also disclosed herewith.

### Definitions

The section headings used herein are for organizational purposes only and are not to be construed as limiting the subject matter described.

As used herein, the term “DXM-MVL” or “DXM-MVLs” refers to a multivesicular liposome composition encapsulating dexmedetomidine. In some embodiments, the composition is a pharmaceutical formulation, where the dexmedetomidine encapsulated multivesicular liposome particles are suspended in a liquid suspending medium to form a suspension. In some such embodiments, the DXM-MVL suspension may also include free or unencapsulated dexmedetomidine. In some cases, the free or unencapsulated dexmedetomidine may be less than about 10%, 9%, 8%, 7%, 6%, 5%, 4%, 3%, 2%, 1%, 0.5%, 0.2% or 0.1%, by weight of the total amount of the dexmedetomidine in the composition, or in a range defined by any of the two preceeding values.

As used herein, the term “encapsulated” means that dexmedetomidine is inside a liposomal particle, for example, the MVL particles, the unilamellar vesicles (ULVs) or multilamellar vesicles (MLVs). In some instances, dexmedetomidine may also be on an inner surface, or intercalated in a membrane, of the MVLs.

As used herein, the term “unencapsulated dexmedetomidine” or “free dexmedetomidine” refers to dexmedetomidine outside the liposomal particles, for example the MVL, UVL or MLV particles. For example, unencapsulated dexmedetomidine may reside in the suspending solution of these particles.

As used herein, the term “median particle diameter” refers to volume weighted median particle diameter of a suspension.

As used herein, “DepoDXM” refers to dexmedetomidine encapsulated in multivesicular liposomes DepoDXM may be characterized by a packed particle volume (PPV) measured in % (v/v). In some embodiments, such DepoDXM formulations contain from about 10% to about 80% (v/v), from about 15% to about 75% (v/v), or from about 20% to about 70% (v/v), or from about 30% to about 65% (v/v), or from about 40% to about 60% (v/v), multivesicular liposome particles. In one particular embodiment, DepoDXM formulations contain about 50% (v/v) multivesicular liposome particles. In one embodiment, DepoDXM formulations contain about 40% (v/v) multivesicular liposome particles. In another embodiment, DepoDXM formulations contain about 45% (v/v) multivesicular liposome particles. In some embodiments, DepoDXM may be used interchangeably with DXM-MVLs.

As used herein, a “pH adjusting agent” refers to a compound that is capable of modulating the pH of an aqueous phase.

As used herein, the terms “tonicity” and “osmolality” are measures of the osmotic pressure of two solutions, for example, a test sample and water separated by a semi-permeable membrane. Osmotic pressure is the pressure that must be applied to a solution to prevent the inward flow of water across a semi-permeable membrane. Osmotic pressure and tonicity are influenced only by solutes that cannot readily cross the membrane, as only these exert an osmotic pressure. Solutes able to freely cross the membrane do not affect tonicity because they will become equal concentrations on both sides of the membrane. An osmotic pressure provided herein is as measured on a standard laboratory vapor pressure or freezing point osmometer.

As used herein, the term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include sorbitol, glucose, fructose, galactose, mannose, sorbose, ribose, deoxyribose, dextrose, neuraminic acid. An oligosaccharide is a carbohydrate consisting of more than one monomeric saccharide unit connected via glycosidic bond(s) either branched or in a chain. The monomeric saccharide units within an oligosaccharide can be the same or different. Depending on the number of monomeric saccharide units the oligosaccharide is a di-, tri-, tetra-, penta- and so forth saccharide. In contrast to polysaccharides, the monosaccharides and oligosaccharides are water soluble. Examples of oligosaccharides include sucrose, trehalose, lactose, maltose and raffinose.

As used herein, the term “minimal sedation/anxiolsyis” is synonymous with “anxiolysis” and means that the patient may have impaired cognitive function and physical coordination but has a normal response to verbal stimulation and that the patients airways, spontaneous ventilation and cardiovascular function are all unaffected.

As used herein, the term “arousable sedation” is synonymous with the terms “conscious sedation” as well as “moderate sedation/analgesia” and refers to a drug-induced state during which a patient responds purposefully to verbal commands or tactile stimulation. Although cognitive function and physical coordination may be impaired, airway reflexes require no intervention, spontaneous ventilation is adequate and cardiovascular function is maintained. (Note that withdrawal from a painful stimulus is not considered a purposeful response.) Thus, the patient remains asleep but is easily arousable. This state is in contrast to “deep sedation/analgesia”, where the patient gives a purposeful response following repeated or painful stimulation. In addition in the “deep sedation/analgesia” state, airway intervention may be required for the patient, spontaneous ventilation may be inadequate and cardiovascular function is usually maintained. Finally, by way of contrast, none of the three terms are synonymous with the term “general anesthesia”. The latter term refers to a state where the patient is unarousable even with painful stimulus, airway intervention is often required, spontaneous ventilation is frequently inadequate, and cardiovascular function may be impaired. The instant formulations are administered to maintain minimal sedation and arousable sedation but not deep sedation/analgesia or general anesthesia.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

### Multivesicular Liposomes Formulations

MVLs are a group of unique forms of synthetic membrane vesicles that are different from other lipid-based delivery systems such as unilamellar liposomes and multilamellar liposomes (Bangham, et al., J Mol. Bio., 13:238-252, 1965). The main structural difference between multivesicular liposomes and unilamellar liposomes (also known as unilamellar vesicles, “ULVs”), is that multivesicular liposomes contain multiple aqueous chambers per particle. The main structural difference between multivesicular liposomes and multilamellar liposomes (also known as multilamellar vesicles, “MLVs”), is that in multivesicular liposomes the multiple aqueous chambers are non-concentric. Multivesicular liposomes generally have between 100 to 1 million chambers per particle and all the internal chambers are interconnected by shared lipid-bilayer walls that separate the chambers. The structural differences between unilamellar, multilamellar, and multivesicular liposomes are illustrated in Sankaram et al., U.S. Pat. Nos. 5,766,627 and 6,132,766.

The structural and functional characteristics of multivesicular liposomes are not directly predictable from current knowledge of unilamellar vesicles and multilamellar vesicles. Multivesicular liposomes have a very distinctive internal morphology, which may arise as a result of the special method employed in the manufacture. Topologically, multivesicular liposomes are defined as having multiple non-concentric chambers within each particle, resembling a “foam-like” or “honeycomb-like” matrix; whereas multilamellar vesicles contain multiple concentric chambers within each liposome particle, resembling the “layers of an onion.”

The presence of internal membranes distributed as a network throughout multivesicular liposomes may serve to confer increased mechanical strength to the vesicle. The particles themselves can occupy a very large proportion of the total formulation volume. The packed particle volume (PPV) of MVLs which is measured in a manner analogous to a hematocrit, representing the volume of the formulation that the particles make up and can approach as high as 80%. Typically the PPV is about 50%. At 50% PPV, the multivesicular liposome formulation typically consists of less than 5% w/w lipid. Thus, the encapsulated volume is approximately 50% while having a relatively low lipid concentration. The multivesicular nature of multivesicular liposomes also indicates that, unlike for unilamellar vesicles, a single breach in the external membrane of multivesicular vesicles will not result in total release of the internal aqueous contents.

Thus, multivesicular liposomes formulations consist of microscopic, spherical particles composed of numerous nonconcentric aqueous chambers. The individual chambers are separated by lipid bilayer membranes composed of synthetic versions of naturally occurring lipids, resulting in a delivery vehicle that is both biocompatible and biodegradable. Thus, DXM-MVL formulations include microscopic, spherical particles composed of numerous nonconcentric aqueous chambers encapsulating dexmedetomidine for controlled release drug delivery. Such formulation is intended to prolong the local delivery of dexmedetomidine, thereby enhancing the duration of action of the reduction of pain or anxiety, or providing arousable sedation rather than deep sedation. The DXM-MVL formulation or composition provides either local site or systemic sustained delivery, and can be administered by a number of routes including subcutaneous, intra-articular into joints, intramuscular into muscle tissue, intraperitoneal, intrathecal, or application to an open wound, or body cavities such as the nasal cavity.

Some embodiments of the present application relate to multivesicular liposome formulations encapsulating dexmedetomidine, the formulations include encapsulated dexmedetomidine, a lipid component comprising at least one amphipathic lipid and at least one neutral lipid, and one or more pH modifying agents. In some embodiments, the formulation also comprises unencapsulated dexmedetomidine, also known as free dexmedetomidine. For example, the formulation may comprise less than 10%, 5%, 2% or 1% by weight of unencapsulated dexmedetomidine. It is important that the unencapsulated dexmedetomidine is maintained at a level that is sufficiently low to avoid exposure of the patient to doses that induce undesired deep sedation and/or hemodynamic effects. In some embodiments, the DXM-MVL formulation is intended to provide minimum sedation, mild sedation, or arousable sedation in a patient. In some further embodiments, a pharmaceutical composition comprising a DXM-MVL formulation described herein comprises about 500 μg, 450 μg, 400 μg, 350 μg, 300 μg, 250 μg, 200 μg, 150 μg, 100 μg, 90 μg, 80 μg, 70 μg, 60 μg, 50 μg, 40 μg, 30 μg, 20 μg, 10 μg, 5 μg or less of unencapsulated dexmedetomidine. In some further embodiments, such pharmaceutical composition is for a single injection or administration (i.e., a single dose). In one embodiment, the pharmaceutical composition comprises about 50 μg or less of unencapsulated dexmedetomidine for a single injection. A single administration of the pharmaceutical composition may provide sustained release of dexmedetomidine for 2 to 14 days, or 3 to 7 days.

Lipid Components

In some embodiments of the formulations described herein, the lipid components of the MVLs comprise at least one amphipathic lipid and at least one neutral lipid.

A “water-in-oil” type emulsion is formed from two immiscible phases, a lipid phase and a first aqueous phase. The lipid phase is made up of at least one amphipathic lipid and at least one neutral lipid in a volatile organic solvent, and optionally cholesterol and/or cholesterol derivatives. The term “amphipathic lipid” refers to molecules having a hydrophilic “head” group and a hydrophobic “tail” group and may have membrane-forming capability. As used herein, amphipathic lipids include those having a net negative charge, a net positive charge, and zwitterionic lipids (having no net charge at their isoelectric point). The term “neutral lipid” refers to oils or fats that have no vesicle-forming capabilities by themselves, and lack a charged or hydrophilic “head” group. Examples of neutral lipids include, but are not limited to, glycerol esters, glycol esters, tocopherol esters, sterol esters which lack a charged or hydrophilic “head” group, and alkanes and squalenes.

The amphipathic lipid is chosen from a wide range of lipids having a hydrophobic region and a hydrophilic region in the same molecule. Suitable amphipathic lipids include, but are not limited to zwitterionic phospholipids, including phosphatidylcholines, phosphatidylethanolamines, sphingomyelins, lysophosphatidylcholines, and lysophosphatidylethanolamines; anionic amphipathic phospholipids such as phosphatidylglycerols, phosphatidylserines, phosphatidylinositols, phosphatidic acids, and cardiolipins; cationic amphipathic lipids such as acyl trimethylammonium propanes, diacyl dimethylammonium propanes, stearylamine, and the like. Non-limiting exemplary phosphatidyl cholines include dioleyl phosphatidyl choline (DOPC), dierucoyl phosphatidyl choline or 1,2-dierucoyl-sn-glycero-3-phosphocholine (DEPC), 1,2-didecanoyl-sn-glycero-3-phosphocholine (DDPC), 1,2-dilinoleoyl-sn-glycero-3-phosphocholine (DLOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1-myristoyl-2-palmitoyl-sn-glycero 3-phosphocholine (MPPC), 1-myristoyl-2-stearoyl-sn-glycero-3-phosphocholine (MSPC), 1-palmitoyl-2-myristoyl-sn-glycero-3-phosphocholine (PMPC), 1-palmitoyl-2-oleoyl-sn-glycero-3-phosphocholine (POPC), 1-palmitoyl-2-stearoyl-sn-glycero-3-phosphocholine (PSPC), 1-stearoyl-2-myristoyl-sn-glycero-3-phosphocholine (SMPC), 1-stearoyl-2-oleoyl-sn-glycero-3-phosphocholine (SOPC), or 1-stearoyl-2-palmitoyl-sn-glycero-3-phosphocholine (SPPC). Non-limiting examples of phosphatidyl glycerols include dipalmitoylphosphatidylglycerol or 1,2-dipalmitoyl-sn-glycero-3-phospho-rac-(1-glycerol) (DPPG), 1,2-dierucoyl-sn-glycero-3-phospho-rac-(1-glycerol) (DEPG), 1,2-dilauroyl-sn-glycero-3-phospho-rac-(1-glycerol) (DLPG), 1,2-dimyristoyl-sn-glycero-3-phospho-rac-(1-glycerol) (DMPG), 1,2-dioleoyl-sn-glycero-3-phospho-rac-(1-glycerol) (DOPG), 1,2-distearoyl-sn-glycero-3-phospho-rac-(1-glycerol) (DSPG), 1-palmitoyl-2-oleoyl-sn-glycero-3-phospho-rac-(1-glycerol) (POPG), or salts thereof, for example, the corresponding sodium salts, ammonium salts, or combinations of the salts thereof.

Suitable neutral lipids include but are not limited to triglycerides, propylene glycol esters, ethylene glycol esters, and squalene. Non-limiting exemplary triglycerides useful in the instant formulations and processes are triolein (TO), tripalmitolein, trimyristolein, trilinolein, tributyrin, tricaproin, tricaprylin (TC), and tricaprin. The fatty chains in the triglycerides useful in the present application can be all the same, or not all the same (mixed chain triglycerides), or all different. Propylene glycol esters can be mixed diesters of caprylic and capric acids.

In some further embodiments, the lipid components contain phosphatidyl choline or salts thereof, phosphatidyl glycerol or salts thereof, and at least one triglyceride. In further embodiments, the phosphatidyl choline and the phosphatidyl glycerol are present in MVLs in a mass ratio of about 10:1 to about 3:1.

In some embodiments, the amphipathic lipid comprises phosphatidylcholine, or phosphatidylglycerol or salts thereof, or combinations thereof. In some embodiments, the phosphatidyl choline is dierucoyl phosphatidyl choline (DEPC). In some embodiments, the phosphatidyl glycerol is dipalmitoyl phosphatidyl glycerol (DPPG). In some embodiments, the phosphatidylcholine is selected from DEPC, DSPC, DMPC, DOPC, or a combination thereof. In further embodiments, the DEPC and the DPPG are present in MVLs in a mass ratio of DEPC:DPPG of about 10:1 to about 1:1, or about 10:1 to about 3:1.

In further embodiments, the neutral lipid comprises triglyceride, propylene glycol ester, ethylene glycol ester, or squalene, or combinations thereof. In some embodiments the neutral lipid comprises triglyceride. In some embodiments the triglyceride comprises triolein or tricaprylin, or a combination thereof. In some further embodiments, the multivesicular liposomes further comprise cholesterol and/or a plant sterol.

pH Modifying Agents

The pH modifying agents that may be used in the present MVL formulations are selected from organic acids, organic bases, inorganic acids, or inorganic bases, or combinations thereof. Suitable inorganic acids (also known as mineral acids) that can be used in the present application include, but are not limited to hydrochloric acid (HCl), sulfuric acid (H₂SO₄), phosphoric acid (H₃PO₄), nitric acid (HNO₃), etc. Suitable organic acids that can be used in the present application include, but are not limited to acetic acid, aspartic acid, citric acid, formic acid, glutamic acid, glucuronic acid, lactic acid, malic acid, tartaric acid, etc. Suitable organic bases that can be used in the present application include, but are not limited to histidine, arginine, lysine, tromethamine (Tris), etc. Suitable inorganic bases that can be used in the present application include, but are not limited to sodium hydroxide, calcium hydroxide, magnesium hydroxide, potassium hydroxide, etc.

In some embodiments, the pH modifying agents are selected from the group consisting of inorganic acids, organic bases, and combinations thereof. In some embodiments, the pH modifying agents are selected from the group consisting of organic acids, organic bases, and combinations thereof. In some embodiments, the inorganic acid is phosphoric acid. In some embodiments, the organic acid is selected from tartaric acid, or glutamic acid, or a combination thereof. In some embodiments, the organic base is selected from histidine, or lysine, or combinations thereof. In some further embodiments, at least one pH modifying agent resides in the first aqueous component of the multivesicular liposomes and said pH modifying agent comprises an inorganic acid, for example, phosphoric acid. In further embodiments, at least one pH modifying agent resides in a second aqueous component used in the process of preparing the multivesicular liposomes, and said pH modifying agent comprises an organic base. In further embodiments the organic base comprises histidine, lysine, or a combination thereof.

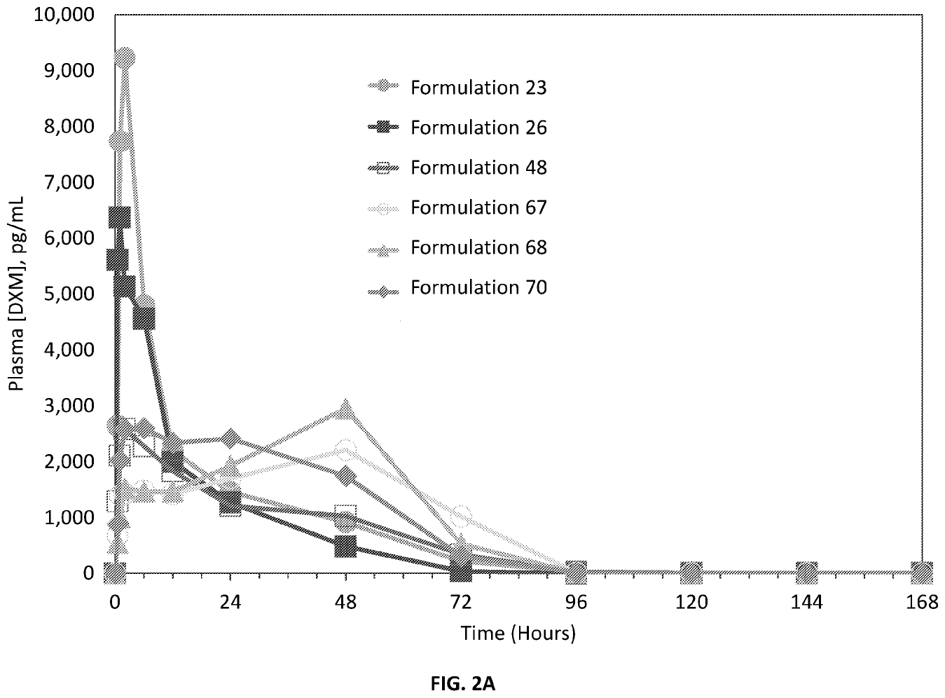

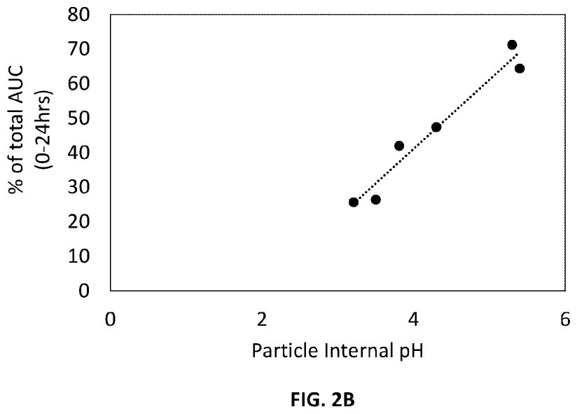

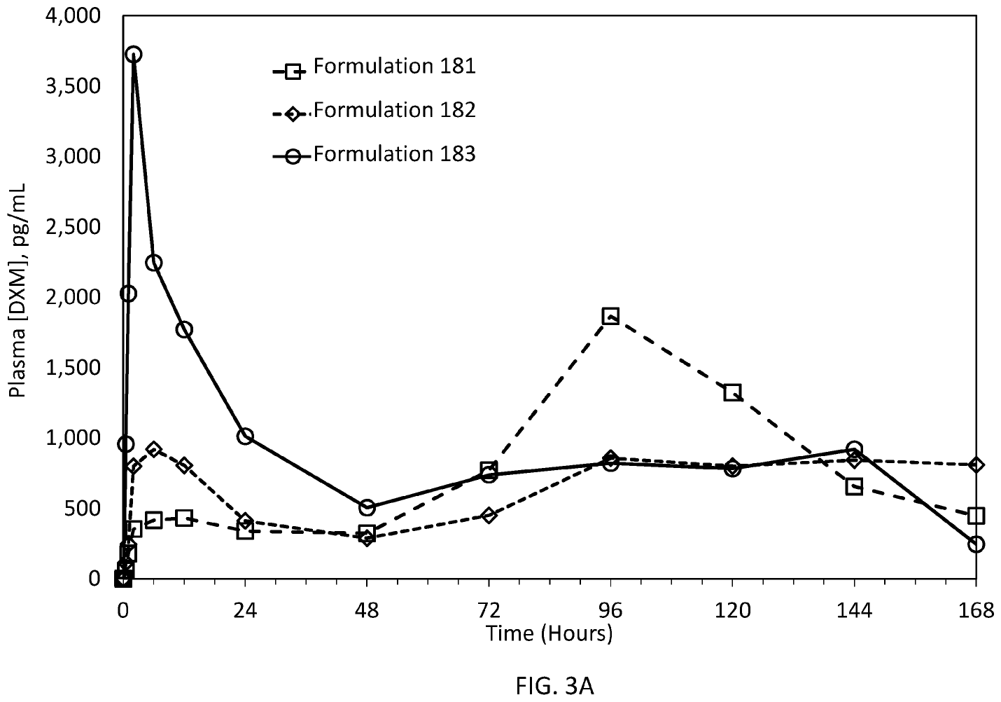

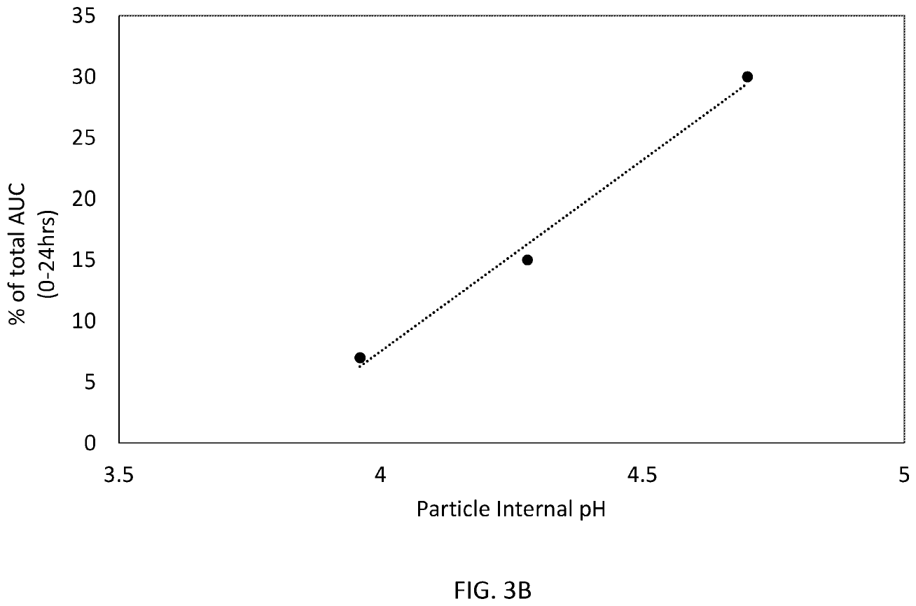

In some embodiments, the internal pH of the MVLs is about 1.0, 1.5, 2.0, 2.5, 3.0, 3.5, 4.0, 4.5, 5.0, 5.5, 6.0, 6.5, 7.0, 7.5, 8.0, 8.5, or 9.0, or within a range defined by any two of the preceding pH values. In some embodiments, the dexmedetomidine encapsulated multivesicular liposomes have an internal pH from about 2.0 to about 8.0, from about 2.5 to about 6.5, from about 3.0 to about 5.5, or from about 3.5 to about 5.0. In further embodiments, the internal pH of the DXM-MVLs has an internal pH from about 3.8 to about 4.8, or about 4.0 to about 4.5. The internal pH of the DXM-MVLs is important for the sustained release rate of the DXM from the MVL particles. It has been observed that when the internal pH of the DXM-MVLs increases from about 3.5 to about 5.5, the % total AUC of DXM release in the first 24 hours increases substantially, for example, from less about 10% to over about 60%. See FIGS. 2B and 3B. However, when the internal pH is too low (for example, less than about 1), it may also cause lipid membrane hydrolysis, which renders the particles very unstable. In some instances, the optimal internal pH of the DXM-MVLs provides less than about 20%, 15%, 10%, or 5% release of the DXM in the first 24 hours. In some such embodiments, the % release of DXM is measured by % total AUC of DXM.

In some embodiments of the formulations described herein, the MVL particles are suspended in a suspending solution. The suspending solution may comprise one or more pH modifying agents, and/or may perform a buffering function. The suspending solution defines the external pH of the MVL formulation. In some embodiments, the pH of the suspending solution is about 2.5, 3.0, 3.5, 4.0, 4.5, 5.0, 5.5, 6.0, 6.5, 7.0, 7.5, 8.0, 8.5, or 9.0, or within a range defined by any two of the preceding pH values. In some embodiments, the dexmedetomidine encapsulated multivesicular liposomes have an external pH (i.e., the pH of the suspending solution where multivesicular liposome particles reside) from about 4.0 to about 7.5. In some further embodiments, the external pH is from about 3.0 to about 7.0, or from about 5.5 to about 6.8. In some embodiments, the suspending solution is the same as the second aqueous component of the MVLs.

Tonicity Agents

In some embodiments of the formulations described herein, the first aqueous component of the MVLs further comprises one or more tonicity agents. Tonicity agents sometimes are also called osmotic agents. Non-limiting exemplary osmotic agents suitable for the MVL formulation of the present application include monosaccharides (e.g., glucose, and the like), disaccharides (e.g., sucrose and the like), polysaccharide or polyols (e.g., sorbitol, mannitol, Dextran, and the like), or amino acids.

In some embodiments, the one or more tonicity agents may be selected from an amino acid, a sugar, or combinations thereof. In some further embodiments, one or more tonicity agents are selected from dextrose, sorbitol, sucrose, lysine, or combinations thereof.

Particle Sizes

In some embodiments of the formulations described herein, the DXM encapsulated MVL particles have a median particle diameter of about 5, 10, 15, 20, 25, 30, 35, 40, 45, 50, 55, 60, 65, 70, 75, 80, 85, 90, 95, or 100 μm, or within a range defined by any two of the preceding values. In some further embodiments, the multivesicular liposomes have a median particle diameter ranging from about 7 μm to about 40 μm. In some further embodiments, the multivesicular liposomes have a median particle diameter ranging from about 10 μm to about 25 μm. In still some further embodiments, the multivesicular liposomes have a median particle diameter (d50) ranging from about 12 μm to about 18 μm.

In some embodiments, the MVLs may optionally comprise additional therapeutic agent(s). In some other embodiments, DXM is the only therapeutic agent in the MVLs.

In some embodiments, the MVL particles are suspended in a liquid suspending solution or medium. In some further embodiments, the liquid suspending medium is a buffered saline solution. In some such embodiments, the MVL particle suspension has a PPV (%) of about 40%, 45%, 50%, or 55%. In further embodiments, the concentration of dexmedetomidine in the liquid suspension is about 0.05 mg/mL, 0.1 mg/mL, 0.2 mg/mL, 0.5 mg/mL, 1.0 mg/mL, 1.5 mg/mL, 2.0 mg/mL, 2.5 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg/mL, or 30 mg/mL, or in a range defined by any of the two preceding values. In some further embodiments, the concentration of dexmedetomidine in the particle suspension is from about 0.1 mg/mL to about 20 mg/mL, from about 3.5 mg/mL to about 8 mg/mL, or from about 4 mg/mL to about 5 mg/mL.

In any embodiments of the dexmedetomidine multivesicular lipsome formulations described herein, the multivesicular liposomes are stable at 37° C. for at least 2, 3, 4, 5, 6, or 7 days. Furthermore, the formulation may be stable at 5° C. for at least 1 week, 2 weeks, 3 weeks, 4 weeks, 2 months, 3 months, 6 months, 9 months, 12 months, 18 months or 24 months. As used herein, the term “stable” means that the multivesicular liposomes particles in the suspending solution maintain the structural integrity and dexmedetomidine remains encapsulated in the multivesicular liposomes without excessively leaking out of multivesicular lipsomes in free form, during certain storage condition for a period of time. In some embodiments, the DXM-MVL formulations described herein are stable at 5° C. for 6 months with less than about 10%, 9%, 8%, 7%, 6%, 5%, 4%, 3%, 2%, 1%, 0.5%, or 0.1% of dexmedetomidine by weight in the free or unencapsulated form. In some embodiments, the DXM-MVL formulations described herein are stable at 37° C. for 3 days with less than about 30%, 25%, 20%, 15%, 10%, 9%, 8%, 7%, 6%, 5%, 4%, 3%, 2%, or 1% of dexmedetomidine by weight in the free or unencapsulated form.

### Methods of Treatment

Some embodiments of the present application are related to methods for treating, ameliorating or preventing pain, anxiety, or the hemodynamic complications of pain and anxiety, or inducing arousable sedation, comprising administering a DXM-MVL pharmaceutical composition, as described herein, to a subject in need thereof. For example, the instant DXM-MVL formulations can be used for pre-surgical medication, in procedural sedation for procedures such as colonoscopy, pediatric patients undergoing tonsillectomy, vitreoretinal surgery, transesophageal echocardiography, awake carotid endarterectomy, shockwave lithotripsy, as an adjuvant in local and regional (e.g., administration by epidural, caudal, or spinal administration) techniques, intra-articular use, controlling hypertension, attenuating the response to tracheal intubation and extubation, as an anesthetic sprating agent, cardiovascular stabilizing effect (e.g. —treating arrhythmias and the deleterious cardiovascular effects of acute cocaine intoxication and overdose); reducing the extent of myocardial ischemia during cardiac surgery; vascular surgery; thoracic surgery; and conventional Coronary Artery Bypass Grafting (CABG); as well was with patients undergoing mitral valve replacement; providing cereboral hemodynamice stability and preventing sudden increase in intracranial pressure during intubation, extubation and head pin insertion; sedating obese patients due to the fact in does not cause respiratory depression; awake intubation; monitored anaesthesia care; post operative analgesia; paediatric use in various intubation and noninvasive procedures; and in the treatment of symptoms of distress (intractable pain, agitation or delirium) at the end of life; spinal surgery; treatment of alcohol or drug withdrawal symptoms, the management of tetanus in the ICU; as an antishivering agent, and in preventing ethanol-induced neurodegeneration.

Further embodiments also include a method for inducing arousable sedation in a subject, comprising administering a pharmaceutical composition described herein to a subject in need thereof.

In some embodiments of the methods described herein, the administration is parenteral. In some further embodiments, the parenteral administration may be selected from the group consisting of subcutaneous injection, tissue injection, intramuscular injection, intraarticular, spinal injection, intraocular injection, epidural injection, intrathecal injection, intraotic injection, perineural injection, and combinations thereof. In particular embodiments, the parenteral administration is subcutaneous injection or tissue injection.

In any of the embodiments, the instant pharmaceutical compositions can be administered by bolus injection, e.g., subcutaneous bolus injection, intramuscular bolus injection, intradermal bolus injection and the like.

Administration of the instant DXM-MVL formulations is accomplished using standard methods and devices, e.g., pens, injector systems, needle and syringe, a subcutaneous injection port delivery system, catheters, and the like.

In some embodiments, the DXM-MVL pharmaceutical composition may be administered every 2, 3, 4, 5, 6, 7, 8, 9, 10, 11, 12, 13 or 14 days. In some such embodiments, the pharmaceutical composition may be administered every 3 to 7 days. The number of administrations may change depending on effectiveness of the dose, observed side effects, desire to titrate up to a desired dose, external factors (e.g., a change in another medication), or the length of time that the dosage form has been administered.

In some embodiments, the DXM-MVL pharmaceutical composition is administered in a dose ranging from about 0.01 μg/kg/h to about 1.5 μg/kg/h, about 0.02 μg/kg/h to about 1.4 μg/kg/h, about 0.03 μg/kg/h to about 1.3 μg/kg/h, about 0.04 μg/kg/h to about 1.2 μg/kg/h, about 0.05 μg/kg/h to about 1.1 μg/kg/h, about 0.1 μg/kg/h to about 1.0 μg/kg/h, from about 0.2 μg/kg/h to about 0.9 μg/kg/h, or from 0.5 μg/kg/h to about 0.8 μg/kg/h. The DXM-MVL pharmaceutical composition is intended to provide minimum sedation, mild sedation, or arousable sedation in a patient. In some embodiments, the DXM-MVL pharmaceutical composition comprises free or unencapsulated dexmedetomidine, for example about 500 μg, 450 μg, 400 μg, 350 μg, 300 μg, 250 μg, 200 μg, 150 μg, 100 μg, 90 μg, 80 μg, 70 μg, 60 μg, 50 μg, 40 μg, 30 μg, 20 μg, 10 μg, 5 μg or less of unencapsulated dexmedetomidine. It is preferred that no more than 200 μg, 150 μg, 100 μg, or 50 μg of unencapsulated DXM be delivered in a single administration of the DXM-MVL. In some embodiments, a single dose of the DXM-MVL pharmaceutical composition comprises about 3.5 mg, 3.0 mg, 2.5 mg, 2.0 mg, 1.5 mg, 1.0 mg, or 0.5 mg of dexmedetomidine. In one embodiment, a single dose of the DXM-MVL pharmaceutical composition comprises about 2.5 mg of dexmedetomidine. In some embodiments, a single dose of the DXM-MVL pharmaceutical composition comprises about 2 ml, 1.5 ml. 1.0 ml or 0.5 ml of the composition in volume. In some further embodiments, the amount of dexmedetomidine delivered in a single injection during 2-day period is from about 0.3 mg to about 5.0 mg, from about 0.65 mg to about 4.0 mg, or from about 1.65 mg to about 3.4 mg. In some further embodiments, the amount of dexmedetomidine delivered in a single injection during 14-day period is from about 2.4 mg to about 35.3 mg, from about 4.7 mg to about 28.2 mg, or from about 11.8 mg to about 23.5 mg.

### Methods of Manufacturing

Some embodiments of the present application relate to a process for preparing dexmedetomidine encapsulated multivesicular liposomes, the process comprising: mixing a first aqueous component with a lipid component comprising at least one organic solvent, at least one amphipathic lipid, and at least one neutral lipid to form a first water-in-oil emulsion, wherein at least one of the first aqueous component and the lipid component comprises dexmedetomidine; combining the first water-in-oil emulsion with a second aqueous component to form a second emulsion; and substantially removing the organic solvent from the second emulsion to form multivesicular liposomes.

In some embodiments, the process further includes diluting the second emulsion in a third aqueous solution prior to substantially removing the organic solvent. In some embodiments, the process further includes isolating the multivesicular liposome particles and suspending them in a liquid suspending medium (e.g., a buffered saline solution) to form a suspension of multivesicular liposomes.

In some embodiments of the process described herein, the organic solvent is substantially removed by exposing the second emulsion to a gas atmosphere. Organic solvent may be removed by blowing a gas over the second emulsion, or sparging gas in the second emulsion, or spraying the second emulsion into a chamber with a continuous stream of circulating gas.

In some embodiments of the process described herein, the first aqueous component comprises dexmedetomidine and at least one pH modifying agent. In some embodiments, the pH modifying agent of the first aqueous component is an inorganic acid, an organic acid, an inorganic base, or an organic base, or combinations thereof. In some such embodiments, the pH modifying agent is phosphoric acid. In some other embodiments, the pH modifying agent is selected from histidine or lysine. In some embodiments, the first aqueous component may also include one or more osmotic agents. The osmotic agent may be selected from a saccharide, such as sucrose. In some such embodiments, the volume of the lipid component is greater than the volume of the first aqueous component. In some other embodiments of the process described herein, dexmedetomidine is incorporated into the lipid component. In some such embodiments, the volume of the lipid component is the same or substantially the same as the volume of the first aqueous component, for example, the volume of the lipid component and the volume of the first aqueous component is about 1:1.

In some embodiments of the process described herein, the pH range of the first aqueous component is about 1.0, 1.5, 2.0, 2.5, 3.0, 3.5, 4.0, 4.5, 5.0, 5.5, 6.0 or 6.5, or a range defined by any two of proceeding values. In some further embodiments, the pH range of the first aqueous component is from about 1.0 to about 6.0, or from about 2.0 to about 5. In certain cases, it was observed that when the pH level was high in the first aqueous component, the encapsulated DXM was more likely to leak out of the MVLs. In contrast, lower pH level in the first aqueous component renders the finished product more stable at higher storing temperatures (for example, room temperature or 37° C.).

As described herein, the internal pH of the final DXM-MVLs is important for the sustained release profile of the DXM. During the manufacturing process, the internal pH of the final product may be controlled by the pH of first aqueous component, where DXM is mixed with one or more pH adjusting agents. In some further embodiments, the molar ratio of the DXM and the pH adjusting agent(s) in the first aqueous component is about 1:1, 1:1.1, 1:1.2, 1:1.3, 1:1.4, 1:1.5, 1:1.6, 1:1.7, 1:1.8, 1:1.9, 1:2, 1:2.5, 1:3, 1:3.5, 1:4, 1:4.5, 1:5, 1:5.5, 1:6, 1:6.5, 1:7, 1:7.5, 1:8, 1:9 or 1:10. In further embodiments, the ratio of DXM to the pH adjustment agent is between about 1:1.4 to 1:1.6, or about 1:1.5, when the DXM loading solution is about 10 mg/mL (50 mM). In one embodiment, the pH adjusting or modifying agent comprises or is an inorganic acid (e.g., phosphoric acid). When the concentration of free DXM loading solution is decreased, increased amount of unencapsulated DXM has been observed in the final product (see Table 4 described herein) possibly due to increased internal pH. As described herein, it is important that the unencapsulated dexmedetomidine is maintained at a level that is sufficiently low to avoid exposure of the patient to DXM doses that induce undesired deep sedation and/or hemodynamic effects. It was surprisingly discovered that when a lower concentration of free DXM loading solution (e.g., 1 mg/mL or 0.5 mg/mL) is used in the lipid solution component, unencapsulated/free DXM maybe suppressed by increasing the acid to DXM ratio.

In some embodiments of the process described herein, the osmolality of the first aqueous component of the MVLs is about 260, 270, 280, 290, 300, 310, 320, 330, 340, or 350 mOsm/kg, or within a range defined by any two of the preceding values. In some further embodiments, the osmolality of the first aqueous component of the MVLs is from about 250 mOsm/kg to about 350 mOsm/kg, or from about 280 mOsm/kg to 310 mOsm/kg.

In some embodiments of the process described herein, the second aqueous component comprises at least one pH modifying agent and at least one tonicity agent. In some such embodiments, the tonicity agent comprises sorbitol, sucrose, or dextrose, or combinations thereof. In some embodiments, the osmolality of the second aqueous component is about 80, 90, 100, 110, 120, 130, 140, 150, 160, 170, 180, 190, 200, 210, 220, 230, 240, 250, 260, 270, 280, 290, 300, 350, 400, 450, or 500 mOsm/kg, or in a range defined by any two of the preceding values. In some embodiments, the osmolality of the second aqueous component is from about 150 mOsm/kg to about 190 mOsm/kg, from about 160 mOsm/kg to about 180 mOsm/kg, or from about 165 mOsm/kg to about 175 mOsm/kg. In one embodiment, the osmolality of the second aqueous component is about 173 mOsm/kg.

In some embodiments of the process described herein, the pH range of the second aqueous component is about 5.0, 5.5, 6.0, 6.5, 7.0, 7.5, 8.0, 8.5, 9.0, 9.5, 10, 10.5, 11, 11.5, or 12 or in a range defined by any two of the preceding values. In some such embodiments, the pH range of the second aqueous component is from about 6.0 to about 11.5, or from about 7.0 to about 11.

After the organic solvent is removed, the resulting multivesicular liposome particles are diluted, centrifuged and the supernatant is replaced with saline, optionally containing one or more buffering agents (e.g. 20 mM sodium phosphate at pH from 5.5 to 7.6, for example at pH 6.8 or 7). After washing, the MVL particles were diluted in saline or other buffer solutions to yield the final product as a liquid suspension with about 50% or about 45% packed particle volume (PPV). In some such embodiments, the concentration of encapsulated dexmedetomidine in the suspension is from about 0.2 mg/mL to about 10 mg/mL, from about 0.5 mg/mL to about 9 mg/mL, from about 1 mg/mL to about 8 mg/mL, from about 2 mg/mL to about 6 mg/mL, or from about 3 mg/mL to about 5 mg/mL. In some such embodiments, the unencapsulated or free DXM is about 10%, 9%, 8%, 7%, 6%, 5%, 4%, 3%, 2% or less by weight of total amount of dexmedetomidine in the suspension. In some embodiments, the concentration of unencapsulated DXM in the final product suspension is less than about 1 mg/mL, 0.9 mg/mL, 0.8 mg/mL, 0.7 mg/mL, 0.6 mg/mL, 0.5 mg/mL, 0.4 mg/mL, 0.3 mg/mL, 0.2 mg/mL, 0.1 mg/mL, 0.05 mg/mL or 0.01 mg/mL.

Some further embodiments of the present disclosure include dexmedetomidine encapsulated multivesicular liposomes prepared by the process described herein.

In some embodiments of the process described herein, the lipid components contain phosphatidyl choline or salts thereof, phosphatidyl glycerol or salts thereof, and at least one triglyceride. In some embodiments, the amphipathic lipid comprises phosphatidylcholine, or phosphatidylglycerol or salts thereof, or combinations thereof. In some embodiments, the phosphatidyl choline is dierucoyl phosphatidyl choline (DEPC). In some embodiments, the phosphatidyl glycerol is dipalmitoyl phosphatidyl glycerol (DPPG). In some embodiments, the phosphatidylcholine is selected from DEPC, DSPC, DMPC, DOPC, or a combination thereof. In further embodiments, the neutral lipid comprises triglyceride, propylene glycol ester, ethylene glycol ester, or squalene, or combinations thereof. In some embodiments the neutral lipid comprises triglyceride. In some embodiments the triglyceride comprises triolein or tricaprylin, or a combination thereof. In some further embodiments, the multivesicular liposomes further comprise cholesterol and/or a plant sterol.

The concentrations of the amphipathic lipids, neutral lipids, and cholesterol present in the water-immiscible solvent used to make the MVLs typically range from 1-120 mM, 2-120 mM, and 10-120 mM, respectively. In some embodiments, the concentrations of the amphipathic lipids, neutral lipids, and cholesterol may range from about 20 mM to about 80 mM, about 8 mM to about 80 mM, and about 25 to about 80 mM, respectively. Specific examples of such concentrations are summarized in Tables A1-A3 herein.

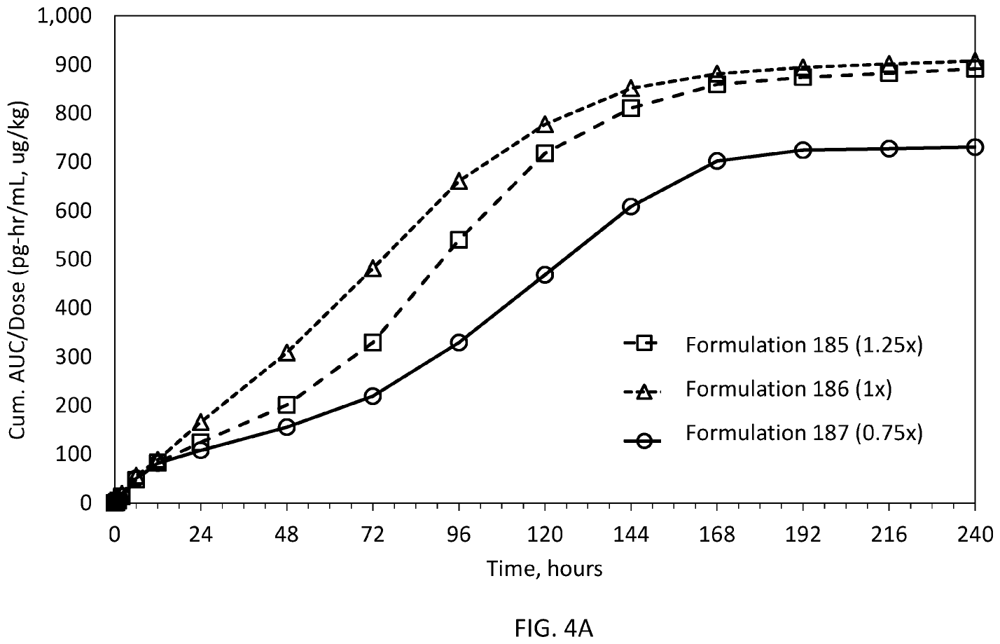

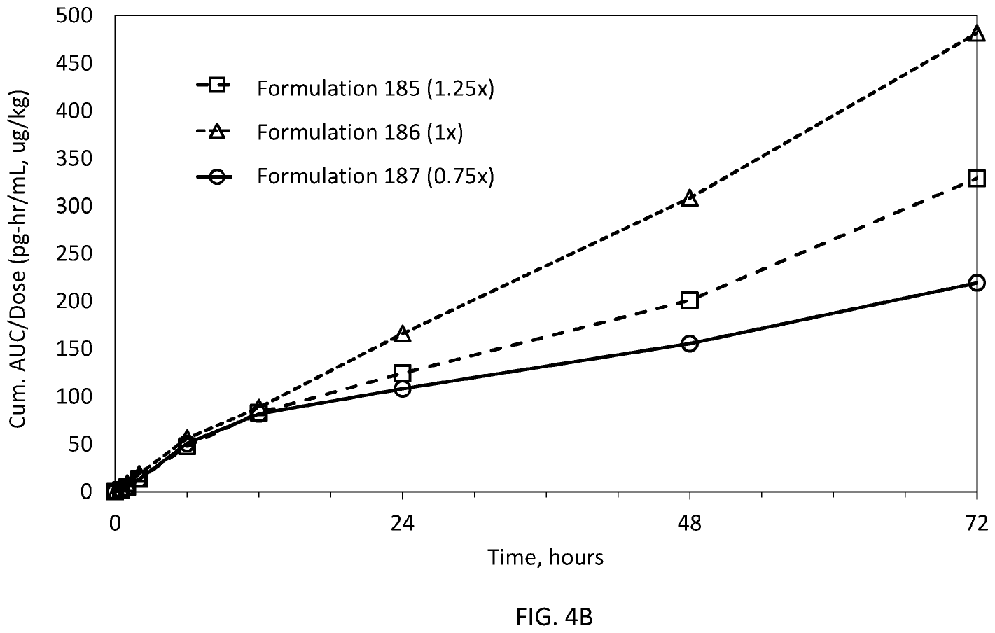

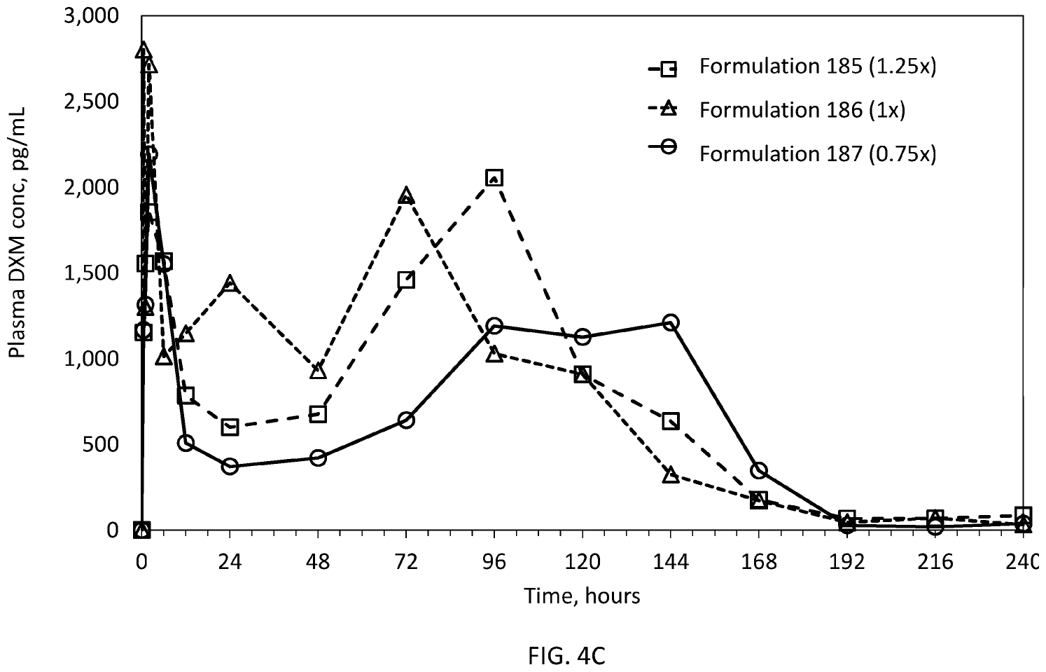

In some embodiments, adjusting the concentration of certain lipid component(s) may have an impact on the sustained release rate of DXM. While it is generally understood that when a higher concentration of the lipid component(s) are used in the manufacturing process of the MVLs, a slower release of the active agent may be observed, at least partially due to the improved strength of the lipid membrane of the MVL particles. However, high lipid concentrations may also have certain drawbacks, such as difficulty in handling of the lipid mixture due to increased stickiness and clogging of the pores of the filter during the filtration of the MVL particles. In some examples, the DXM-MVLs comprise DPPG. When DPPG concentration is decreased to 75% (0.75×) of the standard DPPG concentration, an improved DXM release profile was surprisingly observed in the first 72 hours (see, for example, FIG. 4B, comparing Formulation 186 to Formulation 187). In some such embodiments, the concentrations of the amphipathic lipids (such as phosphotidylcholine or phosphotidylgycerol or salts thereof) in the water-immiscible solvent used to make the MVLs range from about 5 mM to about 20 mM, from about 5 mM to about 15 mM, or from about 8 mM to about 11 mM. In further embodiments, the concentrations of DPPG in the water-immiscible solvent used to make the MVLs range from about 5 mM to about 20 mM, from about 5 mM to about 15 mM, or from about 8 mM to about 11 mM.

In some further embodiments, the concentration of certain lipid components may also affect the internal particle pH of the final DXM-MVL product. In some instances, when the concentration of DPPG is decreased, it causes a decrease in the internal particle pH of the final product.

Many types of volatile organic solvents can be used in the present application, including ethers, esters, halogenated ethers, hydrocarbons, halohydrocarbons, or freon. For example, diethyl ether, chloroform, methylene chloride, tetrahydrofuran, ethyl acetate, and any combinations thereof are suitable for use in making the formulations. In some embodiments, methylene chloride is used. In some other embodiments, chloroform is used.

The lipid component and first aqueous component are mixed by mechanical turbulence, such as through use of rotating or vibrating blades, shaking, extrusion through baffled structures or porous pipes, or by ultrasound, or by the use of a three fluid nozzle (described in U.S. Pat. No. 9,737,482) to produce a water-in-oil emulsion. The water-in-oil emulsion can then be dispersed into a second aqueous component by means described above, to form solvent-containing spherules suspended in the second aqueous component, a water-in-oil-in-water emulsion is formed. The term “solvent-containing spherules” refers to a microscopic spheroid droplet containing organic solvent, within which are suspended multiple smaller droplets of aqueous solution.

The volatile organic solvent is then removed from the spherules by exposing to a pressurized stream of gas. For instance, such a pressurized stream of gas can cause surface evaporation from the second emulsion, sparging the second emulsion with a gas, or contacting the second emulsion with a gas in a spray chamber. When the solvent is substantially or completely evaporated, MVLs are formed. Gases which can be used for the evaporation include nitrogen, argon, helium, oxygen, hydrogen, and carbon dioxide, mixtures thereof, or clean compressed air. Alternately, the volatile solvent can be removed by sparging, rotary evaporation, diafiltration or with the use of solvent selective membranes, or contacting with a gas in a spray chamber.

As discussed above, DXM can be incorporated in the MVL by inclusion in the first aqueous component. DXM can also be incorporated in the MVLs by inclusion in the lipid component or both the lipid and first aqueous component. The amount of DXM recovered in the instant MVLs was assayed by diluting the suspension of the DXM-MVL 50 fold into 100% methanol, then injecting the resulting mixture into an HPLC (Hewlett-Packard Model 1100 with C-18 column; running solvent system: 51% MeOH; 49% aqueous buffer containing monobasic sodium phosphate (NaH₂PO₄), H₃PO₄, TEA and sodium dodecyl sulfate (“SDS”); pH=2.5) as described in the United States Pharmacopeia 37 (USP 37) assay for organic impurities with some minor modification. In some embodiments, the percent DXM yield is from about 40% to about 90% of the starting DXM amount, more preferably from about 50% to about 90%, more preferably from about 60% to about 90%.

Standard preparation of multivesicular liposomes is illustrated in U.S. Pat. Nos. 5,766,627 and 6,132,766, each of which is incorporated by reference in its entirety. Alternatively, DXM can be remotely loaded to the blank MVL particles, which is described in U.S. Pat. No. 9,974,744.

### Pharmaceutical Compositions

In some embodiments, the MVL formulations of the present application optionally include a pharmaceutically acceptable carrier. The term “pharmaceutically-acceptable carrier”, as used herein, means one or more compatible solid or liquid filler diluents or encapsulating substances, which are suitable for administration to an organism (such as a mammal, e.g., human being) and does not abrogate the biological activity of the active ingredient(s). The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; malt; gelatin; talc; calcium sulfate; polyols such as propylene glycol, glycerine, sorbitol, mannitol, and polyethylene glycol; alginic acid; emulsifiers, such as the TWEENS; salts, such as sodium chloride; wetting agents, such sodium lauryl sulfate; coloring agents; flavoring agents; stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

Effective injectable compositions containing these compounds may be in either suspension or solution form. In the solution form, DXM-MVLs may be diluted in a physiologically acceptable vehicle. Such vehicles comprise a suitable solvent, a tonicity agent such as sucrose or saline, preservatives such as benzyl alcohol, if needed, and buffers. Useful solvents include, for example, water and aqueous alcohols, glycols, and carbonate esters such as diethyl carbonate.

Injectable suspension compositions require a liquid suspending medium, with or without adjuvants, as a vehicle. The suspending medium can be, for example, aqueous solutions of sodium chloride, sucrose, polyvinylpyrrolidone, polyethylene glycol, or combinations of the above.

Suitable physiologically acceptable storage solution components are used to keep the compound suspended in suspension compositions. The storage solution components can be chosen from thickeners such as carboxymethylcellulose, polyvinylpyrrolidone, gelatin and the alginates. Many surfactants are also useful as suspending agents. The suspending medium could also contain lecithin, alkylphenol polyethylene oxide adducts, naphthalenesulfonates, alkylbenzenesulfonates, or the polyoxyethylene sorbitan esters. The DXM-MVL storage suspension solution can contain additional additive(s).

Many substances which affect the hydrophilicity, density, and surface tension of the liquid suspending medium can assist in making injectable suspensions in individual cases. For example, silicone antifoams, sorbitol, and sugars can be useful suspending agents.

In some embodiments, the pharmaceutical composition containing DXM-MVLs as described herein provides sustained release of DXM over 12 hours, over 24 hours, over 36 hours, over 48 hours, over 60 hours, over 72 hours, over 96 hours, over 120 hours, over 144 hours, or over 168 hours. In further embodiments, the pharmaceutical composition provides sustained release of DXM over at least 72 hours (3 days). In still further embodiments, the pharmaceutical composition provides sustained release of DXM over at least 120 hours (5 days). In further embodiments, the pharmaceutical composition provides sustained release of DXM between 5 days to 7 days.

In some embodiments, the pharmaceutical composition containing DXM-MVLs as described herein provides less than about 5%, 10%, 15%, 20%, 25%, or 30% release of DXM in the first 24 hours. In some further embodiments, the pharmaceutical composition containing DXM-MVLs as described herein provides less than about 5%, 10%, 15%, 20%, 25%, or 30% release of DXM in the first 48 hours. In some embodiments, the pharmaceutical composition containing DXM-MVLs as described herein provides less than about 5%, 10%, 15%, 20%, 25%, or 30% release of DXM in the first 72 hours. In some such embodiments, the % release is measured by % total AUC or cumulative AUC of the DXM.

### Examples

The following examples, including experiments and results achieved, are provided for illustrative purposes only and are not to be construed as limiting the present application.

### Example 1: Preparation of DXM-MVL Formulations

DXM-MVL formulations were manufactured as follows: DXM was solubilized in either 1) a 1st aqueous solution containing phosphoric acid and sucrose or 2) an organic dichloromethane solution containing: DOPC, DMPC, DSPC or DEPC, DPPG, with tricaprylin and/or triolein, and cholesterol. Next, the aqueous solution was emulsified with the organic solution resulting in a water-in-oil (W/O) emulsion. The W/O emulsion was then emulsified in a second aqueous solution containing lysine or histidine and sorbitol or dextrose to produce a water-in-oil-in-water (W/O/W) emulsion. The W/O/W emulsion was then diluted with a third aqueous solution containing lysine or histidine and sorbitol or dextrose. This was stirred at 23° C. under a nitrogen stream to remove dichloromethane (DCM) via evaporation. The resulting particles were then diluted in saline, centrifuged, and the supernatant was replaced with saline+/−buffering agents (e.g. 20 mM sodium phosphate at pH's 5.5-7.6). to yield a product with a ˜50% packed particle volume.

During the experiments, it has been observed that when dexmedetomidine was dissolved in the first aqueous component, and an equal volume of the first aqueous component and the lipid component were used to prepare the water-in-oil emulsion (first emulsion), the phase of the emulsion reversed and resulted in an oil-in-water emulsion instead. To circumvent the phase reversion, a higher volume of lipid component solution was used. Alternatively, dexmedetomidine was incorporated in the lipid solution instead of the aqueous solution without requiring an increase in the lipid:aqueous volume ratio.

In addition, certain DXM-MVL formulations manufactured using DXM-containing aqueous solutions at about 290 mOsm resulted in higher free DXM concentrations and more aggregated particles. To reduce free DXM concentrations, an increased mixing speed was used to prepare the first emulsion but failed to address the aggregation challenge. Surprisingly, it was discovered that when using a hypotonic second aqueous component (for example, about 150 mOsm to 180 mOsm, which created an osmotic gradient across the MVL membranes), aggregation was reduced while maintaining desired product attributes. In addition, the drug loading and percent yield were also improved.

Exemplary manufacturing condition and DXM-MVL formulation assay results are summarized in Tables A1-A3 herein.

DXM yield in the instant MVLs was assayed by diluting the suspension of the DXM-MVLs 50 fold into 100% methanol, then injecting the resulting mixture into an HPLC (Hewlett-Packard Model 1100 with C-18 column; mobile phase solvent system: 51% MeOH; 49% aqueous buffer containing NaH₂PO₄, H₃PO₄, TEA and SDS; pH=2.5) as described below in the USP 37 assay for organic impurities with some minor modification. In Tables A1-A3, the following abbreviations are used:
CFM is Chloroform (CFM);
DCM is Dichloromethane (CH₂Cl₂);
Lys is Lysine;
His is Histidine;
Osm/D refers to Osmotic/Density Modifying Agent;
EXP is comprised of DEPC (1,2-dierucoyl-sn-glycero-3-phosphocholine, 20 mM, 17.78 mg/mL); DPPG (1,2-dipalmitoyl-sn-glycero-3-phospho-rac-(1-glycerol), 3.54 mM, 2.64 mg/mL); cholesterol (26.72 mM, 10.34 mg/mL); TC (tricaprylin, 9 mM, 4.32 mg/mL); and water (0.07%).
OBLT is comprised of DEPC (1,2-dierucoyl-sn-glycero-3-phosphocholine, 26 mM, 23.71 mg/mL); DPPG (1,2-dipalmitoyl-sn-glycero-3-phospho-rac-(1-glycerol), 11 mM, 8.34 mg/mL); cholesterol (40 mM, 15.48 mg/mL); TC (tricaprylin, 40 mM, 18.84 mg/mL); and water (0.39%).
Total DXM concentration refers to the amount of encapsulated dexmedetomidine in the multivesicular liposomes and the unencapsulated dexmedetomidine in the liquid suspending medium.
Percent DXM Yield refers to the amount of DXM obtained in the final product particle suspension, as compared to the amount incorporated into either the first aqueous or lipid solutions.
Sup [DXM] is the measurement of unencapsulated DXM concentration in the saline solution used to store DXM-MVL particle suspensions. Prior to measurement, the saline solution was added to the final formulation suspension and allowed to equilibrate over night.
% PPV means packed particle volumes, measured by spinning the suspensions down with a centrifuge and measuring the height of the particles in a lipocrit tube with a ruler.
% Free—the amount of unencapsulated DXM in the supernatant versus the total amount of DXM in the suspension.
[DXM] Total at 45% PPV—The concentration of DXM in the suspension after normalizing the % PPV to 45%.
PSD—particle size distribution.
Suc—Sucrose.
Sorb—Sorbitol.
Dex—Dextrose.
Lys—Lysine.
Hist—Histidine.

The following abbreviations are used in Table B:
The erucic acid concentration indicates the amount of hydrolysis of the DEPC lipid in the formulation. NMT means not more than. Storage sol means normal saline.

From the results demonstrated in Tables A1-A3, certain trends were observed. For example, in some embodiments increasing the first emulsion mixing time caused decreases in unecapsulated DXM and particle size. In some instances, lowering the pH of the first aqueous layer caused an increase in the potency of the dose and also lowered unecapsulated DXM. In some other instances, reducing the second aqueous layer tonicity (forming a hypotonic solution) caused the reduction of the amount of unencapsulated DXM and increased particle size, and increased DXM concentration in the first aqueous layer also led to improved yield and potency of the DXM-MVLs produced. In some instances, incorporating DXM in the lipid component during the preparation of the first emulsion, resulted in higher potency, higher yield and lower D90 particle size, as compared to incorporating DXM in the first aqueous layer. In some cases, formulations with increasing concentrations of triolein had better yields.

### Example 2—Stability Studies of DXM-MVL Formulations

Stability studies were conducted on the DXM-MVL formulations described in Example 1. The numbers of each formulation in Tables A1-A3 herein correspond to those same formulation numbers in which stability studies results are summarized in Table B herein. The conditions for these stability studies are as follows:

Following manufacture, each new formulation is aliquotted into glass pharmaceutical vials. The aliquots are stoppered and stored at 37° C. and 5° C. The vials stored at 37° C. are assayed after 3 days and 7 days to determine the stability of the DXM-MVL formulation suspensions under elevated-temperature conditions. Stability under elevated-temperature conditions has been shown to be predictive of 5° C. stability for MVL-based products. The aliquots stored at 5° C. are assayed after much longer-term storage (for instance, 3 mo, 6 mo, 12 mo).

### Example 3—Pharmacokinetic Studies of DXM-MVL Formulations in Rats

Pharmacokinetic studies of the subcutaneous dosing of the DXM PK studies discussed herein were performed in rats where bolus DXM was compared to various formulations of DXM-MVLs at doses between 0.21-0.42 mg/kg. Female Sprague Dawley rats supplied by Absorption Systems weighing about 310 g received subcutaneous injections of either a bolus of unencapsulate (free or unencapsulated) DXM dissolved in H₃PO₄ solution or one the of the DXM-MVL formulations suspended in saline or buffered saline (20 mM phosphate). Injections were made on the left lateral hind limb of each rat using 100 μL syringes fitted with 25G needles. Each treatment group contained 3 rats.

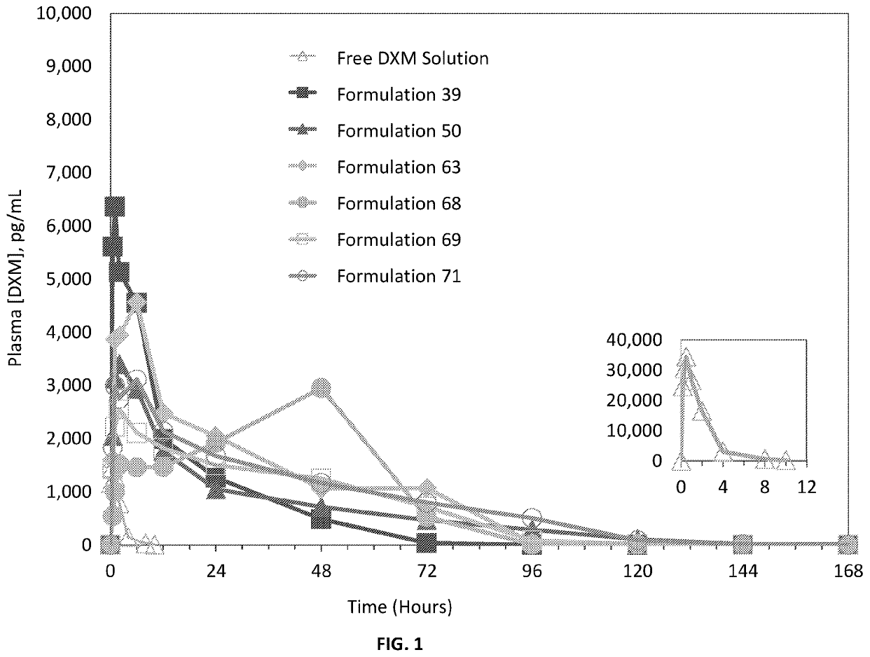

Plasma samples were collected at different times points (0.5, 1, 2, 6, 12, 24, 48, 72 & 96 hour post dose) for analysis. Blood samples were collected via the right saphenous vein using a 19 gauge needle prick or cardiac puncture for the final time point, placed into chilled tubes containing the appropriate anticoagulant, inverted several times to mix, protected from light, and kept on ice until centrifugation. A summary of the data in FIGS. 1 and 2A-2B is set forth below in Tables 1a and 2a below.

FIG. 1 is a line chart illustrating the dose normalized dexmedetomidine plasma levels obtained in rats as a function of time, following administration of several dexmedetomidine encapsulated multivesicular liposome (DXM-MVL) formulations (Formulations 39, 50, 63, 68, 69 and 71) as compared to dexmedetomidine in a saline solution.

FIG. 2A is a line chart illustrating the dose normalized dexmedetomidine plasma levels obtained in rats as a function of time, following administration of several DXM-MVL formulations varying internal pH (Formulations 23, 26, 48, 67, 68 and 70).

FIG. 2B s a line chart illustrating the amount of drug released, in rats, during the first 24 h (% total AUC) as a function of MVL particle internal pH for various DXM-MVL formulations with varied internal pH (Formulations 23, 26, 48, 67, 68 and 70), as seen in FIG. 2A.

TABLE 1a
Summary of PK Data for Selected DXM-MVL Formulations in Rats

| | | | | | | AUC/Dose | % of total | |
|---|---|---|---|---|---|---|---|---|
| | | Dose | Cmax | Cmax/ | Tmax | (pg-hr/mL/ | AUC | Particle |
| Formulation | Description | (mg/kg) | (pg/mL) | Cavg | (hr) | mg/kg) | (0-24 hrs) | Internal pH |
| n/a | Unencapsulated DXM | 0.01 | 1639 | 2.3 | 0.5 | 383 | N/A | N/A |
| | solution | | | | | | | |
| 22 | high total [lipid] | 0.21 | 7662 | 3.9 | 2 | 604 | 60 | 5.3 |
| 23 | high total [lipid], +0.4 | 0.21 | 9773 | 4.1 | 2 | 642 | 64 | 5.4 |
| | mM TO | | | | | | | |
| 26 | 50/50 mix of high and | 0.2 | 6493 | 3.3 | 1 | 483 | 71 | 5.3 |
| | low lipid formulations | | | | | | | |
| 44 | high total [lipid], | 0.21 | 7270 | 2.8 | 6 | 1057 | 56 | 4.9 |
| | hypotonic 2nd Aqueous | | | | | | | |
| 44 (dosed | high total [lipid], 2x | 0.42 | 11527 | 2.9 | 6 | 843 | 57 | 4.9 |
| twice the | dose of Fomulation 44 | | | | | | | |
| volume) | | | | | | | | |
| 31 | high total [lipid], API in | 0.34 | 4378 | 2.5 | 12 | 578 | 44 | 3.8 |
| | organic | | | | | | | |
| 47 | high total [lipid], +4 mM | 0.29 | 8001 | 3.4 | 6 | 625 | 56 | 4.2 |
| | TO | | | | | | | |
| 48 | high total [lipid], | 0.32 | 4097 | 2.3 | 2 | 461 | 47 | 4.3 |
| | 55 mol % Chol | | | | | | | |
| 49 | high total [lipid], 25% | 0.31 | 4335 | 2.8 | 6 | 473 | 44 | 4.1 |
| | Sub TC w/TO | | | | | | | |
| 50 | high total [lipid], 50% | 0.30 | 5118 | 2.4 | 2 | 498 | 49 | 4.6 |
| | Sub TC w/TO | | | | | | | |
| 55 | high total [lipid] | 0.33 | 3998 | 2.9 | 6 | 356 | 48 | 4.1 |
| 56 | high total [lipid], | 0.30 | 5198 | 2.0 | 1 | 564 | 34 | 4.1 |
| | 60 mol % Chol | | | | | | | |
| 63 | 1.5x high total [lipid] | 0.33 | 7578 | 2.9 | 6 | 740 | 48 | 4.6 |
| 64 | 1.25x high total [lipid] | 0.32 | 5707 | 2.6 | 6 | 749 | 41 | 4.3 |
| 67 | high total [lipid], | 0.29 | 3180 | 1.7 | 48 | 667 | 26 | 3.5 |
| | 5.5/4.5(AQ/Lipid | | | | | | | |
| | Combo) | | | | | | | |
| 68 | high total [lipid], | 0.24 | 3505 | 1.7 | 48 | 719 | 26 | 3.2 |
| | 6/4(AQ/Lipid Combo) | | | | | | | |
| 69 | high total [lipid], low | 0.32 | 4064 | 2.0 | 2 | 562 | 40 | 4.8 |
| | potency | | | | | | | |
| 70 | 50/50 mix of high and | 0.29 | 3769 | 2.3 | 6 | 677 | 42 | 3.8 |
| | low lipid, | | | | | | | |
| | 5.5/4.5(AQ/Lipid | | | | | | | |
| | Combo) | | | | | | | |
| 71 | high total [lipid], 25% | 0.32 | 4947 | 2.4 | 6 | 683 | 40 | 4.8 |
| | Sub DEPC w/DSPC | | | | | | | |

TABLE 2a
Comparison of 0-24 h AUC versus Particle Internal
pH for Selected DXM-MVL Formulations in Rats

| | | AUC/Dose | % of total | |
|---|---|---|---|---|
| | | (pg-hr/mL/ | AUC | Particle |
| Formulation | Description | mg/kg) | (0-24 hrs) | Internal pH |
| 23 | high total | 642 | 64 | 5.4 |
| | [lipid], +0.4 | | | |
| | mM TO | | | |
| 26 | 50/50 mix of | 483 | 71 | 5.3 |
| | high and low | | | |
| | lipid formulations | | | |
| 48 | high total | 461 | 47 | 4.3 |
| | [lipid], | | | |
| | 55 mol % Chol | | | |
| 67 | high total | 667 | 26 | 3.5 |
| | [lipid], | | | |
| | 5.5/4.5(AQ/ | | | |
| | Lipid Combo) | | | |
| 68 | high total | 719 | 26 | 3.2 |
| | [lipid], | | | |
| | 6/4(AQ/Lipid | | | |
| | Combo) | | | |
| 70 | 50/50 mix of | 677 | 42 | 3.8 |
| | high and low | | | |
| | lipid, 5.5/4.5(AQ/ | | | |
| | Lipid Combo) | | | |

As can be seen from the above data, plasma DXM levels in rats receiving bolus free DXM in saline peaked within an hour of administration, dropped significantly by 4 hours, and had a half-life of only 2 hours (FIG. 1 inset graph). In contrast, plasma DXM levels in rats that received DXM-MVL formulations were elevated for up to 96 hours as shown in FIG. 1. The DXM-MVL formulations showed improved bioavailability (AUC/Dose), as compared to the bolus DXM group. These results demonstrated that the instant DXM-MVL formulations provided sustained plasma DXM levels for between 3 to 4 days.

Furthermore, it was observed that the MVL particle internal pH plays an important role in the release of the dexmedetomidine in the first 24 hours. As shown in Table 2a (also FIGS. 2A and 2B), the DXM percent AUC in the first 24 hours changed from over 70% to less than 30% when the internal particle pH was adjusted from 5.4 to 3.2. The results suggests that a low internal pH is important to the release rate of the DXM to facilitate a long duration between doses.

### Example 4—Pharmacokinetic Studies of DXM-MVL Formulations in Dogs

Pharmacokinetic studies of the subcutaneous dosing of the DXM PK studies discussed herein were performed in dogs with various formulations of DXM-MVLs at doses between 0.28-0.36 mg/kg. Male Beagle dogs supplied by Absorption Systems weighing about 8-12 kg received subcutaneous injections of DXM-MVL formulations suspended in saline. Injections were made in the left or right pectoralis descendens of each dog using 1 mL syringes fitted with 25G needles. Each treatment group contained 3 dogs. Plasma samples were collected at different times points (0.5, 1, 2, 6, 12, 24, 48, 72, 96, 120, 144, 168, 192, 216, 240 hour post dose) for analysis. Blood samples were collected via the jugular vein or other suitable vessel using a 19 gauge needle prick, placed into chilled tubes containing the appropriate anticoagulant, inverted several times to mix, protected from light, and kept on ice until centrifugation. The pharmacokinetic study results in dogs are summarized in Tables 1b and 2b below.

FIG. 3A is a line chart illustrating the dose normalized dexmedetomidine plasma levels in dogs as a function of time, following SC administration of several DXM-MVL formulations (Formulations 181, 182 and 183) with varying particle internal pH's. FIG. 3B is a line chart illustrating the amount of drug released, in dogs, during the first 24 h (% total AUC) as a function of MVL particle internal pH for various DXM-MVL formulations, as seen in FIG. 3A. It was observed that the MVL particle internal pH plays an important role in the release of the dexmedetomidine in the first 24 hours. As shown in Table 1b, the DXM percent AUC in the first 24 hours changed from over 30% to less than 10% when the internal particle pH was adjusted from 4.7 to 4.0. The results suggests that the internal pH is very important to the release rate of the DXM to facilitate a long duration between doses. As such, lowering internal pH may aid in extending the release rate of DXM in DXM-MVL formulations.

FIG. 4A is a line chart illustrating the cumulative AUC as a function of time of several DXM-MVL formulations (Formulations 185, 186 and 187) with various lipid concentrations. FIG. 4B is an enlarged portion of the line chart shown in FIG. 4A for the first 72 hours. As shown in Table 2b, MVL particle total lipid concentration influences the release of DXM over time (Cumulative AUC/Dose). As shown in FIG. 4A, a 1.25× total lipid concentrated DXM-MVLs (Formulation 185) released slower over time compared with a standard (1×) total lipid concentrated DXM-MVLs (Formulation 186). However, it was surprisingly discovered that when the lipid concentration was lowered to 75% (0.75×) (Formulation 187), a similar slower release profile of the DXM was also observed (as compared to that of Formulation 186), which was in contrary to the general understanding that decreased lipid concentration usually results in reduced stability of the lipid membrane of the MVL particles formed.

Specifically, this trend is more apparent between the first 12-72 hours post-dose (FIG. 4B). Additionally, the DXM-MVL formulations provided sustained plasma DXM levels between 6-7 days (FIG. 4C) independent of total lipid concentration. These findings are important and can significantly affect scale-up manufacturing practices where in some instances high lipid formulations can foul equipment as well as increase cost of goods.

TABLE 1b
Summary of PK Data for Selected DXM-MVL Formulations in Dogs

| | | | | | | AUC/Dose | % of total | |
|---|---|---|---|---|---|---|---|---|
| | | Dose | Cmax | Cmax/ | Tmax | (pg-hr/mL/ | AUC | Particle |
| Formulation | Description | (mg/kg) | (pg/mL) | Cavg | (hr) | mg/kg) | (0-24 hrs) | Internal pH |
| 181 | 1.25x high total [lipid], | 0.36 | 3373 | 3.4 | 96 | 684 | 7 | 4.0 |
| | 1.6:1 Acid:DXM | | | | | | | |
| 182 | 1.25x high total [lipid], | 0.34 | 1570 | 1.6 | 6 | 545 | 15 | 4.3 |
| | 1.5:1 Acid:DXM | | | | | | | |
| 183 | 1.25x high total [lipid], | 0.30 | 5587 | 3.1 | 2 | 751 | 30 | 4.7 |
| | 1.4:1 Acid:DXM | | | | | | | |
| 185 | 1.25x high total [lipid], | 0.28 | 2827 | 2.4 | 96 | 891 | 14 | 4.37 |
| | 1.5:1 Acid:DXM | | | | | | | |
| 186 | 1x high total [lipid], | 0.32 | 4438 | 2.8 | 0.5 | 907 | 18 | 3.76 |
| | 1.5:1 Acid:DXM | | | | | | | |
| 187 | 0.75x high total [lipid], | 0.31 | 3380 | 2.9 | 2 | 731 | 15 | 4.1 |
| | 1.5:1 Acid:DXM | | | | | | | |

TABLE 2b
Comparison of 0-24 h AUC versus Particle Internal
pH for Selected DXM-MVL Formulations in Dogs

| | | AUC/Dose | % of total | |
|---|---|---|---|---|
| | | (pg-hr/mL/ | AUC | Particle |
| Formulation | Description | mg/kg) | (0-24 hrs) | Internal pH |
| 185 | 1.25x high total | 891 | 14 | 4.4 |
| | [lipid], | | | |
| | 1.5:1 Acid:DXM | | | |
| 186 | 1x high total | 907 | 18 | 3.8 |
| | [lipid], 1.5:1 | | | |
| | Acid:DXM | | | |
| 187 | 0.75x high total | 731 | 15 | 4.1 |
| | [lipid], | | | |
| | 1.5:1 Acid:DXM | | | |

### Example 5—Lipid Concentration and Internal Particle pH Correlation of DXM-MVLs

In this example, the internal particle pHs of DXM-MVL formulations with a higher DPPG concentration (14 mM in the lipid component) were compared to those with lower DPPG concentration (5.3 mM in the lipid component) when various concentrations of phosphoric acid was used in the first aqueous component.

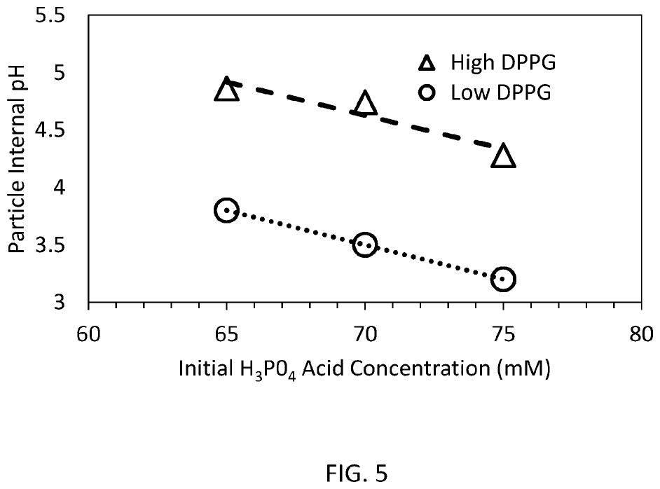

FIG. 5 is a line chart illustrating the internal pH of several DXM-MVL formulations as a function of initial (first aqueous) phosphoric acid concentrations when two different concentrations of dipalmitoylphosphatidylglycerol (DPPG) were used in the DXM-MVL formulations. As shown in FIG. 5, reducing DPPG concentrations results in lower internal particle pH in the final product. It was observed that lower DPPG-containing MVLs (5.3 mM) resulted in lower particle internal pH measurements relative to higher DPPG-containing DXM-MVL formulations (14 mM) with the same amount of initial acid in the first aqueous component. To circumvent this, the initial phosphoric acid (H₃PO₄) concentration was reduced. As particle internal pH may affect the release rate of DXM, modifying DPPG concentrations in DXM-MVL formulations may also help to regulate DXM release profile.

TABLE 3a
Summary of High DPPG Study Formulations

| | Initial H₃PO₄ | Partical |
|---|---|---|
| Formulation | (mM) | Internal pH |
| 184 | 65 | 4.9 |
| 183 | 70 | 4.7 |
| 182 | 75 | 4.3 |

TABLE 3b
Summary of Low DPPG Study Formulations

| | Initial H₃PO₄ | Partical |
|---|---|---|
| Formulation | (mM) | Internal pH |
| 221 | 65 | 3.8 |
| 222 | 70 | 3.5 |
| 223 | 75 | 3.3 |

### Example 6—Potency of Free DXM Loading Solution and Acid to DXM Studies

In this example, the correlation between the final uncapsulated dexmedetomidine and the potency of an initial free DXM loading solution in the first aqueous component was explored. In Table 4, it was observed that in formulations with a fixed acid to drug ratio (1.5:1), decreasing potency of the free DXM solution in the first aqueous component resulted in an increase of the resulting internal pH of the DXM-MVL product and the amount of unencapsulated DXM in the suspension (see Formulations 182, 209 and 210 in Table 4). Surprisingly, when a significantly higher acid to drug ratio was used (5:1) in the first aqueous component, the amount of unencapsulated DXM was substantially reduced even when a lower potency of free DXM loading solution (0.5 mg/mL) was used in the first aqueous component (see Formulation 225 in Table 4).

TABLE 4
Summary of Potency and Free DXM Study Formulations

| | Initial | Initial | | Particle | Free |
|---|---|---|---|---|---|
| | Acid:DXM | DXM | Potency | Internal | DXM |
| Formulation | Ratio | (mg/ml) | (mg/ml) | pH | (%) |
| 182 | 1.5:1 | 10 | 4.1 | 4.3 | 0.5 |
| 209 | 1.5:1 | 1 | 0.25 | 5.9 | 6.4 |
| 210 | 1.5:1 | 0.5 | 0.13 | 6.4 | 5.6 |
| 225 | 5:1 | 0.5 | 0.25 | 4.5 | 0.8 |

TABLE A1

| | FIRST AQUEOUS | | | | | | | | | LIPIDS (no EDTA and | | | |
|---|---|---|---|---|---|---|---|---|---|---|---|---|---|
| | SOLUTION | | | | | | | | | solvent is DCM) | | | |
| | | | | [pH | | | [tonicity | | | | | Base | |
| | Amount | [DXM] | pH | agent] | Ratio | Tonicity | agent] | | | [DXM] | amount | Lipid | [DEPC] |
| # | (mL) | (mg/mL) | modifier | (mM) | acid:DXM | Agent | (mM) | pH | mOsm | (mg/mL) | (mL) | Solution | (mM) |
| 1 | 4.5 | 9.6 | H₃PO₄ | 50 | 1 | Suc | 191 | 4.5 | 298 | 0 | 5.5 | OBLT | 26 |
| 2 | 4.5 | 9.9 | H₃PO₄ | 49 | 1 | Suc | 200 | 4.8 | 304 | 0 | 5.5 | OBLT | 26 |
| 3 | 4.5 | 9.9 | H₃PO₄ | 49 | 1 | Suc | 200 | 4.8 | 304 | 0 | 5.5 | OBLT | 26 |
| 4 | 4.5 | 9.9 | H₃PO₄ | 49 | 1 | Suc | 200 | 4.8 | 304 | 0 | 5.5 | OBLT | 26 |
| 5 | 4.5 | 9.9 | H₃PO₄ | 49 | 1 | Suc | 200 | 4.8 | 304 | 0 | 5.5 | OBLT | 26 |
| 6 | 4.5 | 9.9 | H₃PO₄ | 49 | 1 | Suc | 200 | 4.8 | 304 | 0 | 5.5 | OBLT | 26 |
| 7 | 4.5 | 9.2 | H₃PO₄ | 49 | 1 | Suc | 200 | 4 | 292 | 0 | 5.5 | OBLT | 26 |
| 8 | 4.5 | 9.2 | H₃PO₄ | 49 | 1 | Suc | 200 | 4 | 292 | 0 | 5.5 | OBLT | 26 |
| 9 | 4.5 | 9.2 | H₃PO₄ | 49 | 1 | Suc | 200 | 4 | 292 | 0 | 5.5 | OBLT | 26 |
| 10 | 4.5 | 9.2 | H₃PO₄ | 49 | 1 | Suc | 200 | 4 | 292 | 0 | 5.5 | OBLT | 26 |
| 11 | 4.5 | 9.3 | H₃PO₄ | 48 | 1 | Suc | 206 | 4.1 | 303 | 0 | 5.5 | OBLT | 26 |
| 12 | 4.5 | 9.3 | H₃PO₄ | 48 | 1 | Suc | 206 | 4.1 | 303 | 0 | 5.5 | OBLT | 26 |
| 13 | 4.5 | 9.3 | H₃PO₄ | 48 | 1 | Suc | 206 | 4.1 | 303 | 0 | 5.5 | OBLT | 26 |
| 14 | 4.5 | 9.3 | H₃PO₄ | 48 | 1 | Suc | 206 | 4.1 | 303 | 0 | 5.5 | OBLT | 26 |
| 15 | 4.5 | 9.3 | H₃PO₄ | 48 | 1 | Suc | 206 | 4.1 | 303 | 0 | 5.5 | OBLT | 26 |
| 16 | 4.5 | 9.3 | H₃PO₄ | 48 | 1 | Suc | 206 | 4.1 | 303 | 0 | 5.5 | OBLT | 26 |
| 17 | 4.5 | 9.3 | H₃PO₄ | 48 | 1 | Suc | 206 | 4.1 | 303 | 0 | 5.5 | OBLT | 26 |
| 18 | 4.5 | 9.3 | H₃PO₄ | 48 | 1 | Suc | 206 | 4.1 | 303 | 0 | 5.5 | OBLT | 26 |
| 19 | 4.5 | 9.3 | H₃PO₄ | 48 | 1 | Suc | 206 | 4.1 | 303 | 0 | 5.5 | OBLT | 26 |
| 20 | 4.5 | 9.3 | H₃PO₄ | 48 | 1 | Suc | 206 | 4.1 | 303 | 0 | 5.5 | OBLT | 26 |
| 21 | 4.5 | 9.3 | H₃PO₄ | 48 | 1 | Suc | 206 | 4.1 | 303 | 0 | 5.5 | OBLT | 0 |
| 22 | 4.5 | 9.3 | H₃PO₄ | 48 | 1 | Suc | 206 | 4.1 | 303 | 0 | 5.5 | OBLT | 26 |
| 23 | 4.5 | 9.3 | H₃PO₄ | 48 | 1 | Suc | 206 | 4.1 | 303 | 0 | 5.5 | OBLT | 26 |
| 24 | 4.5 | 9.3 | H₃PO₄ | 48 | 1 | Suc | 206 | 4.1 | 303 | 0 | 5.5 | OBLT | 26 |
| 25 | 4.5 | 9.3 | H₃PO₄ | 48 | 1 | Suc | 206 | 4.1 | 303 | 0 | 5.5 | EXP | 19.8 |
| 26 | 4.5 | 9.3 | H₃PO₄ | 48 | 1 | Suc | 206 | 4.1 | 303 | 0 | 5.5 | EXP/OBLT | 23.1 |
| 27 | 5 | 0 | H₃PO₄ | 70 | N/A | Suc | 178 | 1.8 | 282 | 10 | 5 | OBLT | 26 |
| 28 | 5 | 0 | H₃PO₄ | 70 | N/A | Suc | 178 | 1.8 | 282 | 10 | 5 | OBLT | 26 |
| 29 | 4.5 | 9.5 | H₃PO₄ | 50 | 1 | Suc | 199 | 4 | 296 | 0 | 5.5 | OBLT | 26 |
| 30 | 5 | 10 | H₃PO₄ | 70 | 1.4 | Suc | 178 | 2.5 | 321 | 0 | 5 | OBLT | 26 |
| 31 | 5 | 0 | H₃PO₄ | 70 | N/A | Suc | 178 | 1.8 | 282 | 10 | 5 | OBLT | 26 |
| 32 | 5 | 0 | H₃PO₄ | 70 | N/A | Suc | 178 | 1.7 | 287 | 10 | 5 | OBLT | 26 |
| 33 | 4.5 | 9.5 | H₃PO₄ | 50 | 1 | Suc | 199 | 4 | 296 | 0 | 5.5 | OBLT | 26 |
| 34 | 4.5 | 9.5 | H₃PO₄ | 50 | 1 | Suc | 199 | 4 | 296 | 0 | 5.5 | OBLT | 26 |
| 35 | 4.5 | 9.3 | H₃PO₄ | 57 | 1.2 | Suc | 204 | 3.1 | 303 | 0 | 5.5 | OBLT | 26 |
| 36 | 4.5 | 9.4 | H₃PO₄ | 52 | 1.1 | Suc | 105 | 3.6 | 296 | 0 | 5.5 | OBLT | 26 |
| 37 | 4.5 | 9.3 | H₃PO₄ | 57 | 1.2 | Suc | 204 | 3.1 | 309 | 0 | 5.5 | OBLT | 26 |
| 38 | 4.5 | 9.3 | H₃PO₄ | 57 | 1.2 | Suc | 204 | 3.1 | 309 | 0 | 5.5 | OBLT | 26 |
| 39 | 4.5 | 9.3 | H₃PO₄ | 57 | 1.2 | Suc | 204 | 3.1 | 309 | 0 | 5.5 | OBLT | 26 |
| 40 | 5 | 10 | H₃PO₄ | 70 | 1.4 | Suc | 178 | 1.8 | 282 | 10 | 5 | OBLT | 26 |
| 41 | 5 | 10 | H₃PO₄ | 70 | 1.4 | Suc | 178 | 1.8 | 282 | 10 | 5 | OBLT | 26 |
| 42 | 4.5 | 9.92 | H₃PO₄ | 140 | 2.8 | Suc | 92 | 2.1 | 296 | 0 | 5.5 | OBLT | 26 |
| 43 | 4.5 | 9.92 | H₃PO₄ | 83 | 1.7 | Suc | 168.5 | 2.5 | 305 | 0 | 5.5 | OBLT | 26 |
| 44 | 4.5 | 9.31 | H₃PO₄ | 57 | 1.2 | Suc | 204 | 3.1 | 309 | 0 | 5.5 | OBLT | 26 |
| 45 | 4.5 | 13.45 | H₃PO₄ | 74 | 1.1 | Suc | 167 | 3.1 | 300 | 0 | 5.5 | OBLT | 26 |
| 46 | 4.5 | 14.91 | H₃PO₄ | N/A | N/A | Suc | 156 | 3.3 | 300 | 0 | 5.5 | OBLT | 26 |
| 47 | 5 | 0 | H₃PO₄ | 70 | 1.4 | Suc | 178 | 2 | 290 | 10 | 5 | OBLT | 26 |
| 48 | 5 | 0 | H₃PO₄ | 70 | 1.4 | Suc | 178 | 2 | 290 | 10 | 5 | OBLT | 26 |
| 49 | 5 | 0 | H₃PO₄ | 70 | 1.4 | Suc | 178 | 2 | 290 | 10 | 5 | OBLT | 26 |
| 50 | 5 | 0 | H₃PO₄ | 70 | 1.4 | Suc | 178 | 2 | 290 | 10 | 5 | OBLT | 26 |
| 51 | 4.5 | 0 | H₃PO₄ | 70 | 1.4 | Suc | 178 | 2 | 290 | 10 | 5.5 | OBLT | 0 |
| 52 | 4.5 | 0 | H₃PO₄ | 70 | 1.4 | Suc | 178 | 2 | 290 | 10 | 5.5 | OBLT | 0 |
| 53 | 4.5 | 0 | H₃PO₄ | 70 | 1.4 | Suc | 178 | 2 | 290 | 10 | 5.5 | OBLT | 0 |
| 54 | 4.5 | 0 | H₃PO₄ | 70 | 1.4 | Suc | 178 | 2 | 290 | 10 | 5.5 | OBLT | 0 |
| 55 | 5 | 0 | H₃PO₄ | 70 | 1.4 | Suc | 178 | 2 | 290 | 10 | 5 | OBLT | 26 |
| 56 | 5 | 0 | H₃PO₄ | 70 | 1.4 | Suc | 178 | 2 | 290 | 10 | 5 | OBLT | 26 |
| 57 | 5 | 0 | H₃PO₄ | 50 | 1 | Suc | 178 | 1.8 | 306 | 10 | 5 | OBLT | 26 |
| 58 | 5 | 0 | H₃PO₄ | 55 | 1.1 | Suc | 178 | 1.8 | 305 | 10 | 5 | OBLT | 26 |
| 59 | 5 | 0 | H₃PO₄ | 60 | 1.2 | Suc | 178 | 1.8 | 308 | 10 | 5 | OBLT | 26 |
| 60 | 5 | 0 | H₃PO₄ | 65 | 1.3 | Suc | 178 | 1.8 | 308 | 10 | 5 | OBLT | 26 |
| 61 | 5 | 0 | H₃PO₄ | 70 | 1.4 | Suc | 178 | 2 | 290 | 5 | 5 | OBLT | 26 |
| 62 | 5 | 0 | H₃PO₄ | 70 | 1.4 | Suc | 178 | 2 | 290 | 5 | 5 | OBLT | 26 |
| 63 | 5 | 0 | H₃PO₄ | 70 | 1.4 | Suc | 178 | 2 | 290 | 10 | 5 | OBLT | 39 |
| 64 | 5 | 0 | H₃PO₄ | 70 | 1.4 | Suc | 178 | 2 | 290 | 10 | 5 | OBLT | 33 |
| 65 | 5.5 | 0 | H₃PO₄ | 70 | 1.4 | Suc | 178 | 2 | 290 | 10 | 4.5 | OBLT | 33 |
| 66 | 6 | 0 | H₃PO₄ | 70 | 1.4 | Suc | 178 | 2 | 290 | 10 | 4 | OBLT | 26 |
| 67 | 5.5 | 0 | H₃PO₄ | 70 | 1.4 | Suc | 178 | 2 | 290 | 10 | 4.5 | OBLT | 33 |
| 68 | 6 | 0 | H₃PO₄ | 70 | 1.4 | Suc | 178 | 2 | 290 | 10 | 4 | OBLT | 39 |
| 69 | 5 | 0 | H₃PO₄ | 35 | 1.4 | Suc | 178 | 2 | 290 | 5 | 5 | OBLT | 26 |
| 70 | 5.5 | 0 | H₃PO₄ | 70 | 1.4 | Suc | 178 | 2 | 290 | 10 | 4.5 | OBLT | 37.1 |
| 71 | 4.5 | 0 | H₃PO₄ | 70 | 1.4 | Suc | 178 | 2 | 290 | 10 | 5.5 | OBLT | 19.5 |
| 72 | 5 | 0 | H₃PO₄ | 88 | 1.75 | Suc | 178 | 2 | 290 | 10 | 5 | OBLT | 26 |
| 73 | 5 | 0 | H₃PO₄ | 100 | 2 | Suc | 178 | 2 | 290 | 10 | 5 | OBLT | 26 |
| 74 | 5 | 0 | H₃PO₄ | 70 | 1.4 | Suc | 178 | 2 | 290 | 10 | 5 | OBLT | 29.7 |
| 75 | 5 | 0 | H₃PO₄ | 70 | 1.4 | Suc | 178 | 2 | 290 | 10 | 5 | OBLT | 29.7 |
| 181 | 5 | 0 | H₃PO₄ | 80 | 1.6 | Suc | 170 | N/A | 294 | 10 | 5 | OBLT | 33 |
| | | | | | | | | | | | | 125% | |
| 182 | 5 | 0 | H₃PO₄ | 75 | 1.5 | Suc | 180 | N/A | 290 | 10 | 5 | OBLT | 33 |
| | | | | | | | | | | | | 125% | |
| 183 | 5 | 0 | H₃PO₄ | 70 | 1.4 | Suc | 170 | N/A | 281 | 10 | 5 | OBLT | 33 |
| | | | | | | | | | | | | 125% | |
| 184 | 5 | 0 | H₃PO₄ | 65 | 1.3 | Suc | 180 | N/A | 284 | 10 | 5 | OBLT | 33 |
| | | | | | | | | | | | | 125% | |
| 185 | 5 | 0 | H₃PO₄ | 75 | 1.5 | Suc | 180 | 1.7 | 290 | 10 | 5 | OBLT | 33 |
| | | | | | | | | | | | | 125% | |
| 186 | 5 | 0 | H₃PO₄ | 75 | 1.5 | Suc | 180 | 1.7 | 290 | 10 | 5 | OBLT | 26 |
| | | | | | | | | | | | | 100% | |
| 187 | 5 | 0 | H₃PO₄ | 75 | 1.5 | Suc | 180 | 1.7 | 290 | 10 | 5 | OBLT | 20 |
| | | | | | | | | | | | | 75% | |
| 209 | 5 | 0 | H₃PO₄ | 7.5 | 1.5 | Suc | 183 | N/A | 290 | 1 | 5 | OBLT | 33 |
| | | | | | | | | | | | | 125% | |
| 210 | 5 | 0 | H₃PO₄ | 3.8 | 1.5 | Suc | 184 | N/A | 290 | 0.5 | 5 | OBLT | 33 |
| | | | | | | | | | | | | 125% | |
| 221 | 5 | 0 | H₃PO₄ | 65 | 1.3 | Suc | 186 | N/A | 290 | 10 | 5 | OBLT | 33 |
| | | | | | | | | | | | | 125% | |
| 222 | 5 | 0 | H₃PO₄ | 70 | 1.4 | Suc | 187 | N/A | 290 | 10 | 5 | OBLT | 33 |
| | | | | | | | | | | | | 125% | |
| 223 | 5 | 0 | H₃PO₄ | 75 | 1.5 | Suc | 189 | N/A | 290 | 10 | 5 | OBLT | 33 |
| | | | | | | | | | | | | 125% | |
| 225 | 5 | 0 | H₃PO₄ | 13 | 5 | Suc | 190 | N/A | 290 | 0.5 | 5 | OBLT | 33 |
| | | | | | | | | | | | | 125% | |

| | | | | | | | | SECOND AQUEOUS | | | | | | |
|---|---|---|---|---|---|---|---|---|---|---|---|---|---|---|
| | LIPIDS (no EDTA and | | | | | | | SOLUTION | | | | | | |
| | solvent is DCM) | | | | | | | | | [tonicity | | [pH | | |
| | [DOPC] | DMPC | DSPC | [DPPG] | [chol] | [TC] | [TO] | amount | Tonicity | agent] | pH | agent] | | |
| # | (mM) | mM | mM | (mM) | (mM) | (mM) | (mM) | (mL) | Agent | (mM) | Agent | (mM) | pH | mOsm |
| 1 | 0 | 0 | 0 | 11 | 40 | 40 | 0 | 25 | Sorb | 250 | Lys | 10 | 10 | 263 |
| 2 | 0 | 0 | 0 | 11 | 40 | 40 | 0 | 25 | Suc | 271 | Lys | 10 | 10 | 263 |
| 3 | 0 | 0 | 0 | 11 | 40 | 40 | 0 | 25 | Sorb | 255 | Hist | 10 | 7.8 | 262 |
| 4 | 0 | 0 | 0 | 11 | 40 | 40 | 0 | 25 | Suc | 262 | Hist | 10 | 7.9 | 283 |
| 5 | 0 | 0 | 0 | 11 | 40 | 40 | 0.4 | 25 | Suc | 262 | Hist | 10 | 7.9 | 283 |
| 6 | 0 | 0 | 0 | 11 | 40 | 50 | 0 | 25 | Suc | 262 | Hist | 10 | 7.9 | 283 |
| 7 | 0 | 0 | 0 | 11 | 40 | 40 | 0 | 25 | Suc | 262 | Lys | 10 | 10 | 271 |
| 8 | 0 | 0 | 0 | 11 | 40 | 40 | 0.4 | 25 | Suc | 262 | Lys | 10 | 10 | 271 |
| 9 | 0 | 0 | 0 | 11 | 40 | 40 | 0.8 | 25 | Suc | 262 | Lys | 10 | 10 | 271 |
| 10 | 0 | 0 | 0 | 11 | 40 | 50 | 0 | 25 | Suc | 262 | Lys | 10 | 10 | 271 |
| 11 | 0 | 0 | 0 | 11 | 40 | 40 | 0 | 25 | Sorb | 250 | Lys | 10 | 10 | 263 |
| 12 | 0 | 0 | 0 | 11 | 40 | 40 | 0 | 25 | Sorb | 250 | Lys | 10 | 10 | 263 |
| 13 | 0 | 0 | 0 | 11 | 40 | 40 | 0 | 25 | Sorb | 250 | Lys | 10 | 10 | 263 |
| 14 | 0 | 0 | 0 | 11 | 40 | 40 | 0 | 25 | Sorb | 250 | Lys | 10 | 10 | 263 |
| 15 | 0 | 0 | 0 | 11 | 40 | 40 | 0 | 25 | Sorb | 250 | Lys | 10 | 10 | 263 |
| 16 | 0 | 0 | 0 | 11 | 40 | 40 | 0 | 25 | Sorb | 250 | Lys | 10 | 10 | 263 |
| 17 | 0 | 0 | 0 | 11 | 40 | 40 | 0 | 25 | Sorb | 250 | Lys | 10 | 10 | 263 |
| 18 | 0 | 0 | 0 | 11 | 40 | 40 | 0 | 25 | Sorb | 250 | Lys | 10 | 10 | 263 |
| 19 | 0 | 0 | 0 | 11 | 40 | 40 | 0 | 25 | Sorb | 250 | Lys | 10 | 10 | 263 |
| 20 | 0 | 0 | 0 | 11 | 40 | 40 | 0 | 25 | Sorb | 250 | Lys | 10 | 10 | 263 |
| 21 | 27 | 0 | 0 | 11 | 40 | 40 | 0 | 25 | Sorb | 250 | Lys | 10 | 10 | 263 |
| 22 | 0 | 0 | 0 | 11 | 40 | 40 | 0 | 25 | Sorb | 242 | Lys | 10 | 10 | 264 |
| 23 | 0 | 0 | 0 | 11 | 40 | 40 | 0.4 | 25 | Sorb | 242 | Lys | 10 | 10 | 264 |
| 24 | 0 | 0 | 0 | 11 | 40 | 40 | 0.8 | 25 | Sorb | 242 | Lys | 10 | 10 | 264 |
| 25 | 0 | 0 | 0 | 4 | 27 | 9 | 0 | 25 | Sorb | 242 | Lys | 10 | 10 | 264 |
| 26 | 0 | 0 | 0 | 7 | 34 | 24 | 0 | 25 | Sorb | 242 | Lys | 10 | 10 | 264 |
| 27 | 0 | 0 | 0 | 11 | 40 | 40 | 0 | 24 | Dex | 159 | Lys | 10 | 10 | 173 |
| 28 | 0 | 0 | 0 | 11 | 40 | 40 | 0 | 24 | Dex | 159 | Lys | 10 | 10 | 173 |
| 29 | 0 | 0 | 0 | 11 | 40 | 40 | 0 | 25 | Sorb | 91.4 | Lys | 10 | 10 | 98 |
| 30 | 0 | 0 | 0 | 11 | 40 | 40 | 0 | 24 | Dex | 159 | Lys | 10 | 10 | 173 |
| 31 | 0 | 0 | 0 | 11 | 40 | 40 | 0 | 24 | Dex | 159 | Lys | 10 | 10 | 173 |
| 32 | 0 | 0 | 0 | 11 | 40 | 40 | 0 | 24 | Dex | 159 | Lys | 10 | 10 | 173 |
| 33 | 0 | 0 | 0 | 11 | 40 | 40 | 0 | 25 | Sorb | 91.4 | Lys | 10 | 10 | 98 |
| 34 | 0 | 0 | 0 | 11 | 40 | 40 | 0 | 25 | Sorb | 91.4 | Lys | 10 | 10 | 98 |
| 35 | 0 | 0 | 0 | 11 | 40 | 40 | 0 | 25 | Sorb | 91.4 | Lys | 10 | 10 | 98 |
| 36 | 0 | 0 | 0 | 11 | 40 | 40 | 0 | 25 | Sorb | 91.4 | Lys | 10 | 10 | 98 |
| 37 | 0 | 0 | 0 | 11 | 40 | 40 | 0 | 25 | Sorb | 169 | Lys | 10 | 10 | 173 |
| 38 | 0 | 0 | 0 | 11 | 40 | 40 | 0 | 25 | Sorb | 118 | Lys | 10 | 10 | 126 |
| 39 | 0 | 0 | 0 | 11 | 40 | 40 | 0 | 25 | Sorb | 147 | Lys | 10 | 10 | 149 |
| 40 | 0 | 0 | 0 | 11 | 40 | 40 | 0 | 24 | Dex | 78.4 | Lys | 10 | 10 | 87 |
| 41 | 0 | 0 | 0 | 11 | 40 | 40 | 0 | 24 | Dex | 238 | Lys | 10 | 10 | 258 |
| 42 | 0 | 0 | 0 | 11 | 40 | 40 | 0 | 25 | Sorb | 147 | Lys | 10 | 10 | 149 |
| 43 | 0 | 0 | 0 | 11 | 40 | 40 | 0 | 25 | Sorb | 147 | Lys | 10 | 10 | 149 |
| 44 | 0 | 0 | 0 | 11 | 40 | 40 | 0 | 25 | Sorb | 147 | Lys | 10 | 10 | 149 |
| 45 | 0 | 0 | 0 | 11 | 40 | 40 | 0 | 25 | Sorb | 147 | Lys | 10 | 10 | 149 |
| 46 | 0 | 0 | 0 | 11 | 40 | 40 | 0 | 25 | Sorb | 147 | Lys | 10 | 10 | 149 |
| 47 | 0 | 0 | 0 | 11 | 40 | 40 | 4 | 24 | Dex | 169 | Lys | 10 | 9.7 | 169 |
| 48 | 0 | 0 | 0 | 11 | 46 | 40 | 0 | 24 | Dex | 169 | Lys | 10 | 9.7 | 169 |
| 49 | 0 | 0 | 0 | 11 | 40 | 30 | 10 | 24 | Dex | 169 | Lys | 10 | 9.7 | 169 |
| 50 | 0 | 0 | 0 | 11 | 40 | 20 | 20 | 24 | Dex | 169 | Lys | 10 | 9.7 | 169 |
| 51 | 0 | 0 | 26 | 11 | 40 | 40 | 0 | 24 | Dex | 169 | Lys | 10 | 9.7 | 169 |
| 52 | 0 | 0 | 26 | 11 | 40 | 40 | 4 | 24 | Dex | 169 | Lys | 10 | 9.7 | 169 |
| 53 | 0 | 26 | 0 | 11 | 40 | 40 | 0 | 24 | Dex | 169 | Lys | 10 | 9.7 | 169 |
| 54 | 0 | 26 | 0 | 11 | 40 | 40 | 4 | 24 | Dex | 169 | Lys | 10 | 9.7 | 169 |
| 55 | 0 | 0 | 0 | 11 | 40 | 40 | 0 | 24 | Dex | 169 | Lys | 10 | 9.7 | 169 |
| 56 | 0 | 0 | 0 | 11 | 55 | 40 | 0 | 24 | Dex | 169 | Lys | 10 | 9.7 | 169 |
| 57 | 0 | 0 | 0 | 11 | 70 | 40 | 0 | 24 | Dex | 169 | Lys | 10 | 9.7 | 169 |
| 58 | 0 | 0 | 0 | 11 | 85 | 40 | 0 | 24 | Dex | 169 | Lys | 10 | 9.7 | 169 |
| 59 | 0 | 0 | 0 | 11 | 100 | 40 | 0 | 24 | Dex | 169 | Lys | 10 | 9.7 | 169 |
| 60 | 0 | 0 | 0 | 11 | 115 | 40 | 0 | 24 | Dex | 169 | Lys | 10 | 9.7 | 169 |
| 61 | 0 | 0 | 0 | 11 | 40 | 40 | 0 | 24 | Dex | 169 | Lys | 10 | 9.7 | 169 |
| 62 | 0 | 0 | 0 | 11 | 40 | 40 | 0 | 24 | Dex | 169 | Lys | 10 | 9.7 | 169 |
| 63 | 0 | 0 | 0 | 16.5 | 60 | 60 | 0 | 24 | Dex | 169 | Lys | 10 | 9.7 | 169 |
| 64 | 0 | 0 | 0 | 14 | 50 | 50 | 0 | 24 | Dex | 169 | Lys | 10 | 9.7 | 169 |
| 65 | 0 | 0 | 0 | 14 | 50 | 50 | 0 | 24 | Dex | 169 | Lys | 10 | 9.7 | 169 |
| 66 | 0 | 0 | 0 | 11 | 60 | 40 | 0 | 24 | Dex | 169 | Lys | 10 | 9.7 | 169 |
| 67 | 0 | 0 | 0 | 14 | 50 | 50 | 0 | 24 | Dex | 169 | Lys | 10 | 9.7 | 169 |
| 68 | 0 | 0 | 0 | 16.5 | 60 | 60 | 0 | 24 | Dex | 169 | Lys | 10 | 9.7 | 169 |
| 69 | 0 | 0 | 0 | 11 | 40 | 40 | 0 | 24 | Dex | 169 | Lys | 10 | 9.7 | 169 |
| 70 | 0 | 0 | 0 | 13.9 | 55 | 48.4 | 0 | 24 | Dex | 169 | Lys | 10 | 9.7 | 169 |
| 71 | 0 | 0 | 6.5 | 11 | 40 | 40 | 0 | 25 | Dex | 170 | Lys | 11 | 9.7 | 169 |
| 72 | 0 | 0 | 0 | 11 | 40 | 40 | 0 | 24 | Dex | 170 | Lys | 10 | 9.7 | 169 |
| 73 | 0 | 0 | 0 | 11 | 40 | 40 | 0 | 24 | Dex | 170 | Lys | 10 | 9.7 | 169 |
| 74 | 0 | 0 | 0 | 5.3 | 40.1 | 13.8 | 0 | 24 | Dex | 170 | Lys | 10 | 9.7 | 169 |
| 75 | 0 | 0 | 0 | 5.3 | 40.1 | 28.6 | 0 | 24 | Dex | 170 | Lys | 10 | 9.7 | 169 |
| 181 | 0 | 0 | 0 | 14 | 50 | 50 | 0 | 24 | Dex | 170 | Lys | 10 | 9.7 | 169 |
| 182 | 0 | 0 | 0 | 14 | 50 | 50 | 0 | 24 | Dex | 170 | Lys | 10 | 9.7 | 169 |
| 183 | 0 | 0 | 0 | 14 | 50 | 50 | 0 | 24 | Dex | 170 | Lys | 10 | 9.7 | 169 |
| 184 | 0 | 0 | 0 | 14 | 50 | 50 | 0 | 24 | Dex | 170 | Lys | 10 | 9.7 | 169 |
| 185 | 0 | 0 | 0 | 14 | 50 | 50 | 0 | 24 | Dex | 170 | Lys | 10 | 9.7 | 169 |
| 186 | 0 | 0 | 0 | 11.2 | 40 | 40 | 0 | 24 | Dex | 170 | Lys | 10 | 9.7 | 169 |
| 187 | 0 | 0 | 0 | 8.4 | 30 | 30 | 0 | 24 | Dex | 170 | Lys | 10 | 9.7 | 169 |
| 209 | 0 | 0 | 0 | 14 | 50 | 50 | 0 | 24 | Dex | 170 | Lys | 10 | 9.7 | 169 |
| 210 | 0 | 0 | 0 | 14 | 50 | 50 | 0 | 24 | Dex | 170 | Lys | 10 | 9.7 | 169 |
| 221 | 0 | 0 | 0 | 5.3 | 50 | 50 | 0 | 24 | Dex | 170 | Lys | 10 | 9.7 | 169 |
| 222 | 0 | 0 | 0 | 5.3 | 50 | 50 | 0 | 24 | Dex | 170 | Lys | 10 | 9.7 | 169 |
| 223 | 0 | 0 | 0 | 5.3 | 50 | 50 | 0 | 24 | Dex | 170 | Lys | 10 | 9.7 | 169 |
| 225 | 0 | 0 | 0 | 14 | 50 | 50 | 0 | 24 | Dex | 170 | Lys | 10 | 9.7 | 169 |

TABLE A2

| | THIRD AQUEOUS | | | | | | | |
|---|---|---|---|---|---|---|---|---|
| | SOLUTION | | | | | | | |
| | | | [tonicity | | [pH | | | |
| | Amount | Tonicity | agent] | pH | agent] | | | WASH/ |
| # | (mL) | Agent | [symbol] | Agent | (mM) | pH | mOsm | STORAGE |
| 1 | 100 | Sorb | 250 | Lys | 10 | 10 | 263 | saline |
| 2 | 100 | Suc | 271 | Lys | 10 | 10 | 263 | saline |
| 3 | 100 | Sorb | 255 | Hist | 10 | 7.8 | 262 | saline |
| 4 | 100 | Suc | 262 | Hist | 10 | 7.9 | 283 | saline |
| 5 | 100 | Suc | 262 | Hist | 10 | 7.9 | 283 | saline |
| 6 | 100 | Suc | 262 | Hist | 10 | 7.9 | 283 | saline |
| 7 | 100 | Sorb | 250 | Lys | 10 | 10 | 262 | saline |
| 8 | 100 | Sorb | 250 | Lys | 10 | 10 | 262 | saline |
| 9 | 100 | Sorb | 250 | Lys | 10 | 10 | 262 | saline |
| 10 | 100 | Sorb | 250 | Lys | 10 | 10 | 262 | saline |
| 11 | 100 | Sorb | 250 | Lys | 10 | 10 | 263 | saline |
| 12 | 100 | Sorb | 250 | Lys | 10 | 10 | 263 | saline |
| 13 | 100 | Sorb | 250 | Lys | 10 | 10 | 263 | saline |
| 14 | 100 | Sorb | 250 | Lys | 10 | 10 | 263 | saline |
| 15 | 100 | Sorb | 250 | Lys | 10 | 10 | 263 | saline |
| 16 | 100 | Sorb | 250 | Lys | 10 | 10 | 263 | saline |
| 17 | 100 | Sorb | 250 | Lys | 10 | 10 | 263 | saline |
| 18 | 100 | Sorb | 250 | Lys | 10 | 10 | 263 | saline |
| 19 | 100 | Sorb | 250 | Lys | 10 | 10 | 263 | saline |
| 20 | 100 | Sorb | 250 | Lys | 10 | 10 | 263 | saline |
| 21 | 100 | Sorb | 250 | Lys | 10 | 10 | 263 | saline |
| 22 | 100 | Sorb | 242 | Lys | 10 | 10 | 264 | saline |
| 23 | 100 | Sorb | 242 | Lys | 10 | 10 | 264 | saline |
| 24 | 100 | Sorb | 242 | Lys | 10 | 10 | 264 | saline |
| 25 | 100 | Sorb | 242 | Lys | 10 | 10 | 264 | saline |
| 26 | 100 | Sorb | 242 | Lys | 10 | 10 | 264 | saline |
| 27 | 140 | Dex | 185 | Lys | 12 | 10 | 204 | saline |
| 28 | 140 | Dex | 185 | Lys | 12 | 10 | 204 | saline |
| 29 | 100 | Sorb | 91.4 | Lys | 10 | 10 | 98 | saline |
| 30 | 140 | Dex | 185 | Lys | 12 | 10 | 204 | saline |
| 31 | 140 | Dex | 185 | Lys | 12 | 10 | 204 | saline |
| 32 | 140 | Dex | 185 | Lys | 12 | 10 | 204 | saline |
| 33 | 100 | Sorb | 91.4 | Lys | 10 | 10 | 98 | saline |
| 34 | 100 | Sorb | 91.4 | Lys | 10 | 10 | 98 | saline |
| 35 | 100 | Sorb | 91.4 | Lys | 10 | 10 | 98 | saline |
| 36 | 100 | Sorb | 91.4 | Lys | 10 | 10 | 98 | saline |
| 37 | 100 | Sorb | 163 | Lys | 10 | 10 | 173 | saline |
| 38 | 100 | Sorb | 118 | Lys | 10 | 10 | 126 | saline |
| 39 | 100 | Sorb | 147 | Lys | 10 | 10 | 149 | saline |
| 40 | 140 | Dex | 92 | Lys | 12 | 10 | 103 | saline |
| 41 | 140 | Dex | 274 | Lys | 12 | 9.9 | 285 | saline |
| 42 | 100 | Sorb | 147 | Lys | 10 | 10 | 149 | saline |
| 43 | 100 | Sorb | 147 | Lys | 10 | 10 | 149 | saline |
| 44 | 100 | Sorb | 147 | Lys | 10 | 10 | 149 | saline |
| 45 | 100 | Sorb | 147 | Lys | 10 | 10 | 149 | saline |
| 46 | 100 | Sorb | 147 | Lys | 10 | 10 | 149 | saline |
| 47 | 140 | Dex | 169 | Lys | 10 | 9.7 | 169 | saline |
| 48 | 140 | Dex | 169 | Lys | 10 | 9.7 | 169 | saline |
| 49 | 140 | Dex | 169 | Lys | 10 | 9.7 | 169 | saline |
| 50 | 140 | Dex | 169 | Lys | 10 | 9.7 | 169 | saline |
| 51 | 140 | Dex | 169 | Lys | 10 | 9.7 | 169 | saline |
| 52 | 140 | Dex | 169 | Lys | 10 | 9.7 | 169 | saline |
| 53 | 140 | Dex | 169 | Lys | 10 | 9.7 | 169 | saline |
| 54 | 140 | Dex | 169 | Lys | 10 | 9.7 | 169 | saline |
| 55 | 140 | Dex | 169 | Lys | 10 | 9.7 | 169 | saline |
| 56 | 140 | Dex | 169 | Lys | 10 | 9.7 | 169 | saline |
| 57 | 140 | Dex | 169 | Lys | 10 | 9.7 | 169 | saline |
| 58 | 140 | Dex | 169 | Lys | 10 | 9.7 | 169 | saline |
| 59 | 140 | Dex | 169 | Lys | 10 | 9.7 | 169 | saline |
| 60 | 140 | Dex | 169 | Lys | 10 | 9.7 | 169 | saline |
| 61 | 140 | Dex | 169 | Lys | 10 | 9.7 | 169 | saline |
| 62 | 140 | Dex | 169 | Lys | 10 | 9.7 | 169 | 20P, 6.8 |
| 63 | 140 | Dex | 169 | Lys | 10 | 9.7 | 169 | saline |
| 64 | 140 | Dex | 169 | Lys | 10 | 9.7 | 169 | saline |
| 65 | 140 | Dex | 169 | Lys | 10 | 9.7 | 169 | saline |
| 66 | 140 | Dex | 169 | Lys | 10 | 9.7 | 169 | saline |
| 67 | 140 | Dex | 169 | Lys | 10 | 9.7 | 169 | saline |
| 68 | 140 | Dex | 169 | Lys | 10 | 9.7 | 169 | saline |
| 69 | 140 | Dex | 169 | Lys | 10 | 9.7 | 169 | saline |
| 70 | 140 | Dex | 169 | Lys | 10 | 9.7 | 169 | saline |
| 71 | 140 | Dex | 170 | Lys | 10 | 9.7 | 169 | saline |
| 72 | 140 | Dex | 170 | Lys | 10 | 9.7 | 169 | 20P, 6.8 |
| 73 | 140 | Dex | 170 | Lys | 10 | 9.7 | 169 | 20P, 6.8 |
| 74 | 140 | Dex | 170 | Lys | 10 | 9.7 | 169 | 20P, 6.8 |
| 75 | 140 | Dex | 170 | Lys | 10 | 9.7 | 169 | 20P, 6.8 |
| 181 | 140 | Dex | 170 | Lys | 10 | 9.7 | 169 | saline |
| 182 | 140 | Dex | 170 | Lys | 10 | 9.7 | 169 | saline |
| 183 | 140 | Dex | 170 | Lys | 10 | 9.7 | 169 | saline |
| 184 | 140 | Dex | 170 | Lys | 10 | 9.7 | 169 | saline |
| 185 | 140 | Dex | 170 | Lys | 10 | 9.7 | 169 | saline |
| 186 | 140 | Dex | 170 | Lys | 10 | 9.7 | 169 | saline |
| 187 | 140 | Dex | 170 | Lys | 10 | 9.7 | 169 | saline |
| 209 | 140 | Dex | 170 | Lys | 10 | 9.7 | 169 | saline |
| 210 | 140 | Dex | 170 | Lys | 10 | 9.7 | 169 | saline |
| 221 | 140 | Dex | 170 | Lys | 10 | 9.7 | 169 | saline |
| 222 | 140 | Dex | 170 | Lys | 10 | 9.7 | 169 | saline |
| 223 | 140 | Dex | 170 | Lys | 10 | 9.7 | 169 | saline |
| 225 | 140 | Dex | 170 | Lys | 10 | 9.7 | 169 | saline |

| | FINAL PRODUCT | | | | | | |
|---|---|---|---|---|---|---|---|
| | | amount | | | | | |
| | pellet | storage | total | Percent | sup | | |
| | mass | sol. | [DXM] | DXM | [DXM] | % | % |
| # | (g) | added | (mg/mL) | Yield | (mg/mL) | PPV | free |
| 1 | 5.9 | 5.3 | 2.7 | 69 | 0.19 | 42 | 4.1 |
| 2 | 3.1 | 2.8 | 2.5 | 32 | 0.25 | 42 | 5.9 |
| 3 | 6.1 | 5.5 | 2.3 | 60 | 0.06 | 32 | 1.8 |
| 4 | 4.2 | 3.8 | 2.8 | 50 | 0.04 | 40 | 0.9 |
| 5 | 5.0 | 4.5 | 3.1 | 65 | 0.04 | 37 | 0.8 |
| 6 | 5.2 | 4.7 | 3.0 | 67 | 0.04 | 36 | 0.9 |
| 7 | 3.8 | 3.5 | 2.8 | 49 | 0.08 | 45 | 1.6 |
| 8 | 4.0 | 3.6 | 3.5 | 64 | 0.11 | 51 | 1.5 |
| 9 | 4.6 | 4.1 | 3.3 | 70 | 0.13 | 42 | 2.3 |
| 10 | 3.9 | 3.5 | 2.9 | 51 | 0.08 | 45 | 1.5 |
| 11 | 5.1 | 4.6 | 2.8 | 64 | 0.09 | 44 | 1.8 |
| 12 | 5.0 | 4.5 | 2.5 | 56 | 0.08 | 48 | 1.7 |
| 13 | 6.1 | 5.5 | 2.2 | 60 | 0.09 | 47 | 2.2 |
| 14 | 5.3 | 4.8 | 2.1 | 51 | 0.13 | 45 | 3.4 |
| 15 | 5.8 | 5.2 | 2.3 | 60 | 0.10 | 48 | 2.3 |
| 16 | 4.5 | 4.0 | 2.5 | 50 | 0.20 | 45 | 4.5 |
| 17 | 5.9 | 5.3 | 2.2 | 60 | 0.12 | 48 | 2.8 |
| 18 | 4.9 | 4.4 | 2.3 | 51 | 0.21 | 46 | 4.9 |
| 19 | 5.8 | 5.3 | 2.2 | 57 | 0.07 | 46 | 1.8 |
| 20 | 5.2 | 4.7 | 2.4 | 57 | 0.06 | 49 | 1.4 |
| 21 | 5.4 | 4.9 | 2.7 | 66 | 0.11 | 42 | 2.4 |
| 22 | 5.3 | 4.8 | 2.6 | 64 | 0.07 | 47 | 1.4 |
| 23 | 5.1 | 4.6 | 2.7 | 63 | 0.07 | 47 | 1.4 |
| 24 | 5.6 | 5.1 | 2.8 | 71 | 0.15 | 44 | 3.0 |
| 25 | 5.3 | 4.7 | 2.7 | 64 | 0.24 | 38 | 5.5 |
| 26 | 6.1 | 5.5 | 2.6 | 71 | 0.09 | 45 | 1.9 |
| 27 | 6.0 | 5.2 | 4.6 | 103 | 0.05 | 48 | 0.6 |
| 28 | 5.9 | 5.2 | 3.5 | 78 | 0.07 | 47 | 1.1 |
| 29 | 6.0 | 5.4 | 2.6 | 69 | 0.30 | 41 | 6.7 |
| 30 | | Reversed | Reversed | Reversed | Reversed | Reversed | Reversed |
| 31 | 6.2 | 5.2 | 4.2 | 96 | 0.05 | 49 | 0.6 |
| 32 | 6.2 | 5.2 | 4.1 | 94 | 0.06 | 49 | 0.7 |
| 33 | 6.1 | 5.5 | 2.7 | 73 | 0.23 | 41 | 5.0 |
| 34 | 6.0 | 5.4 | 2.7 | 72 | 0.22 | 40 | 4.9 |
| 35 | 6.1 | 5.5 | 2.8 | 78 | 0.28 | 41 | 5.9 |
| 36 | 6.1 | 5.5 | 2.9 | 79 | 0.24 | 41 | 4.8 |
| 37 | 5.9 | 5.3 | 2.8 | 74 | 0.07 | 44 | 1.4 |
| 38 | 6.7 | 6.0 | 2.8 | 85 | 0.07 | 43 | 1.4 |
| 39 | 6.8 | 6.1 | 2.8 | 85 | 0.06 | 45 | 1.2 |
| 40 | 5.3 | 5.0 | 3.6 | 74 | 0.3 | 39.4 | 4.7 |
| 41 | 5.7 | 5.0 | 4.1 | 87 | 0.1 | 44.6 | 1.2 |
| 42 | 5.7 | 5.1 | 3.5 | 84 | 0.06 | 45 | 1.0 |
| 43 | 6.5 | 5.8 | 3.2 | 87 | 0.03 | 44 | 0.5 |
| 44 | 5.8 | 5.5 | 2.6 | 71 | 0.09 | 45 | 1.9 |
| 45 | 6.1 | 5.8 | 3.8 | 75 | 0.12 | 44 | 1.8 |
| 46 | 6.4 | 6.1 | 4.1 | 77 | 0.13 | 43 | 1.8 |
| 47 | 6.5 | 5.8 | 3.7 | 91 | 0.16 | 43.5 | 2.5 |
| 48 | 6 | 5.4 | 4.0 | 92 | 0.06 | 44.2 | 0.9 |
| 49 | 6.2 | 5.6 | 3.9 | 92 | 0.15 | 44.9 | 2.2 |
| 50 | 6.3 | 5.6 | 3.8 | 90 | 0.20 | 44.9 | 2.9 |
| 51 | 4 | 3.6 | 3.0 | 41 | 0.23 | 38.8 | 4.8 |
| 52 | 4.1 | 3.7 | 3.0 | 43 | 0.26 | 36.8 | 5.5 |
| 53 | 1 | 0.5 | Pellet was | | | | |
| | | | too small to | | | | |
| | | | warrant | | | | |
| | | | performing | | | | |
| | | | analyses | | | | |
| 54 | 1.2 | 1 | Pellet was | | | | |
| | | | too small to | | | | |
| | | | warrant | | | | |
| | | | performing | | | | |
| | | | analyses | | | | |
| 55 | 6.1 | 5.5 | 4.2 | 97 | 0.18 | 44.8 | 2.4 |
| 56 | 6.5 | 5.8 | 3.7 | 91 | 0.12 | 44.6 | 1.8 |
| 57 | 6 | 5.4 | 3.3 | 76 | 0.078 | 47.7 | 1.2 |
| 58 | 5.2 | 4.7 | 3.4 | 67 | 0.081 | 43.9 | 1.4 |
| 59 | 5.4 | 4.9 | 3.7 | 75 | 0.075 | 46.1 | 1.1 |
| 60 | 5.6 | 5 | 3.8 | 80 | 0.069 | 44.6 | 1.0 |
| 61 | 2.7 | 2.4 | 4 | 82 | 0.076 | 43 | 1.1 |
| 62 | 2.8 | 2.5 | 4.2 | 88 | 0.045 | 46.9 | 0.6 |
| 63 | 5.2 | 4.6 | 4.2 | 82 | 0.048 | 44.6 | 0.6 |
| 64 | 5.7 | 5.1 | 4 | 87 | 0.053 | 43.7 | 0.7 |
| 65 | 5.7 | 5.1 | 3.5 | 83 | 0.038 | 43.1 | 0.6 |
| 66 | 6.8 | 6.1 | 2.6 | 85 | 0.03 | 46.9 | 0.6 |
| 67 | 5.9 | 5.3 | 3.6 | 90 | 0.05 | 45.4 | 0.7 |
| 68 | 6.2 | 5.6 | 3 | 89 | 0.04 | 44.8 | 0.8 |
| 69 | 5.1 | 4.5 | 2 | 77 | 0.03 | 45.4 | 0.7 |
| 70 | 5.7 | 5.2 | 3.7 | 89 | 0.04 | 44.8 | 0.6 |
| 71 | 5.1 | 4.6 | 4 | 71 | 0.15 | 42.2 | 2.1 |
| 72 | 5.7 | 5.4 | 4.1 | 92 | 0.01 | 42.3 | 0.1 |
| 73 | 5.4 | 4.9 | 4.1 | 85 | 0.01 | 41.8 | 0.1 |
| 74 | 5.9 | 5.3 | 3.8 | 85 | 0.06 | 41.4 | 0.9 |
| 75 | 6.3 | 5.7 | 3.7 | 89 | 0.01 | 42.6 | 0.2 |
| 181 | 5.5 | 4.9 | 4.3 | 90 | 0.07 | 45.4 | 0.8 |
| 182 | 5.8 | 5.1 | 4.1 | 89 | 0.04 | 43.1 | 0.5 |
| 183 | 4.9 | 4.4 | 4.1 | 76 | 0.06 | 44.6 | 0.8 |
| 184 | 5.4 | 4.9 | 3.6 | 74 | 0.06 | 43.1 | 0.9 |
| 185 | 5.5 | 4.9 | 3.3 | 69 | 0.06 | 44 | 1 |
| 186 | 6.1 | 5.5 | 3.8 | 89 | 0.06 | 44 | 0.9 |
| 187 | 5.7 | 5.1 | 3.7 | 80 | 0.09 | 44 | 1.3 |
| 209 | 5.2 | 4.7 | 0.25 | 49 | 0.031 | 49.2 | 6.4 |
| 210 | 6.6 | 5.9 | 0.13 | 65 | 0.014 | 47 | 5.6 |
| 221 | 6.2 | 5.5 | 3.4 | 80 | 0.026 | 46.1 | 0.4 |
| 222 | 6.4 | 5.7 | 3.4 | 81 | 0.033 | 46.1 | 0.5 |
| 223 | 6.3 | 5.7 | 3.4 | 81 | 0.031 | 46.1 | 0.5 |
| 225 | 4.1 | 3.5 | 0.25 | 76 | 0.004 | 48.6 | 0.8 |

[symbol]

indicates data missing or illegible when filed

TABLE A3

| | | | | | | | | | free |
|---|---|---|---|---|---|---|---|---|---|
| | | | [DXM] | | | | | | DXM |
| | | | total at | | | | | | (μg) in |
| | | | 45% PPV | | | PSD (μm) | | | a 2.5 |
| # | % PPV | % free | (mg/mL) | Ext pH | Int pH | d10 | d50 | d90 | mg dose |
| 1 | 42 | 4.1 | 2.9 | 6.5 | 5.2 | 8 | 14 | 63 | 104 |
| 2 | 42 | 5.9 | 2.6 | 6.5 | 5.4 | 7 | 13 | 256 | 147 |
| 3 | 32 | 1.8 | 3.2 | 6.5 | 5.2 | 13 | 235 | 478 | 45 |
| 4 | 40 | 0.9 | 3.2 | 6.6 | 5.2 | 11 | 99 | 699 | 21 |
| 5 | 37 | 0.8 | 3.7 | 6.6 | 5.2 | 10 | 144 | 322 | 21 |
| 6 | 36 | 0.9 | 3.8 | 6.6 | 5.2 | 9.4 | 89 | 324 | 21 |
| 7 | 45 | 1.6 | 2.8 | 6.5 | 5.2 | 7 | 14 | 204 | 39 |
| 8 | 51 | 1.5 | 3.1 | 6.5 | 5.2 | 7 | 14 | 140 | 39 |
| 9 | 42 | 2.3 | 3.6 | 6.5 | 5.2 | 8 | 74 | 336 | 57 |
| 10 | 45 | 1.5 | 2.9 | 6.5 | 5.4 | 7 | 14 | 180 | 38 |
| 11 | 44 | 1.8 | 2.8 | 6.5 | 5.6 | 10 | 62 | 631 | 46 |
| 12 | 48 | 1.7 | 2.3 | 6.5 | 5.5 | 7.9 | 25.5 | 510 | 42 |
| 13 | 47 | 2.2 | 2.1 | 6.5 | 5.4 | 7 | 14 | 208 | 54 |
| 14 | 45 | 3.4 | 2.1 | 6.5 | 5.3 | 8 | 32 | 305 | 85 |
| 15 | 48 | 2.3 | 2.1 | 6.5 | 5.5 | 6 | 40 | 479 | 57 |
| 16 | 45 | 4.5 | 2.5 | 6.5 | N/A | 8 | 19 | 194 | 111 |
| 17 | 48 | 2.8 | 2.1 | 6.5 | N/A | 7 | 61 | 357 | 70 |
| 18 | 46 | 4.9 | 2.3 | 6.6 | N/A | 7 | 18 | 183 | 123 |
| 19 | 46 | 1.8 | 2.1 | 6.6 | N/A | 6 | 12 | 166 | 45 |
| 20 | 49 | 1.4 | 2.2 | 6.6 | N/A | 7 | 12 | 150 | 34 |
| 21 | 42 | 2.4 | 2.9 | 6.5 | N/A | 10 | 28 | 248 | 60 |
| 22 | 47 | 1.4 | 2.5 | 6.5 | 5.3 | 7 | 12 | 194 | 35 |
| 23 | 47 | 1.4 | 2.6 | 6.5 | 5.4 | 7 | 12 | 203 | 35 |
| 24 | 44 | 3.0 | 2.8 | 6.5 | 5.4 | 9 | 170 | 406 | 76 |
| 25 | 38 | 5.5 | 3.2 | 6.5 | 5.6 | 14 | 176 | 472 | 139 |
| 26 | 45 | 1.9 | 2.6 | 6.5 | 5.3 | 7 | 15 | 258 | 48 |
| 27 | 48 | 0.6 | 4.3 | 6 | 4.4 | 7 | 12 | 19 | 14 |
| 28 | 47 | 1.1 | 3.4 | 5.8 | 4.2 | 8 | 13 | 54 | 26 |
| 29 | 41 | 6.7 | 2.8 | 6.2 | 5.1 | 8 | 14 | 58 | 169 |
| 30 | Reversed | Reversed | Reversed | Reversed | Reversed | Reversed | Reversed | Reversed | N/A |
| 31 | 49 | 0.6 | 3.9 | 5.5 | 3.8 | 8 | 14 | 26 | 15 |
| 32 | 49 | 0.7 | 3.8 | 5.2 | 3.5 | 7 | 13 | 25 | 19 |
| 33 | 41 | 5.0 | 3.0 | 6.3 | 5.1 | 9 | 16 | 74 | 126 |
| 34 | 40 | 4.9 | 3.0 | 6.3 | 5.2 | 9 | 17 | 89 | 123 |
| 35 | 41 | 5.9 | 3.1 | 6.1 | 5 | 7.3 | 14.1 | 96 | 147 |
| 36 | 41 | 4.8 | 3.2 | 6.3 | 5.1 | 7.1 | 12.8 | 77.4 | 121 |
| 37 | 44 | 1.4 | 2.8 | 6.9 | 5.1 | 6.4 | 11.4 | 121 | 35 |
| 38 | 43 | 1.4 | 2.9 | 6.6 | 5.1 | 6.6 | 11.5 | 124.1 | 36 |
| 39 | 45 | 1.2 | 2.8 | 6.8 | 5.2 | 6.4 | 11.5 | 244.1 | 30 |
| 40 | 39 | 4.7 | 4.1 | 5.2 | 3.8 | 7.8 | 13.8 | 28.5 | 118 |
| 41 | 44.6 | 1.2 | 4.1 | 5.5 | 3.6 | 7.7 | 13.9 | 129.3 | 30 |
| 42 | 45 | 1.0 | 3.5 | 4.6 | 2.4 | 8.2 | 16.1 | 316.5 | 24 |
| 43 | 44 | 0.5 | 3.2 | 5.7 | 3.4 | 7.5 | 13.4 | 74.1 | 13 |
| 44 | 45 | 1.9 | 2.7 | 6.5 | 4.9 | 6.4 | 11.4 | 102 | 47 |
| 45 | 44 | 1.8 | 3.9 | 6.6 | 5 | 7 | 12.3 | 110.8 | 44 |
| 46 | 43 | 1.8 | 4.3 | 6.7 | 5.1 | 7.8 | 15.3 | 107.4 | 45 |
| 47 | 43.5 | 2.5 | 3.8 | 5.6 | 4.2 | 7.8 | 12.9 | 23.6 | 62 |
| 48 | 44.2 | 0.9 | 4.1 | 6.2 | 4.3 | 7.6 | 13.2 | 43.9 | 23 |
| 49 | 44.9 | 2.2 | 3.9 | 5.7 | 4.1 | 7.9 | 13.1 | 23.3 | 54 |
| 50 | 44.9 | 2.9 | 3.8 | 6 | 4.6 | 7.9 | 13.2 | 23.8 | 73 |
| 51 | 38.8 | 4.8 | 3.5 | 6.4 | 5.4 | 10.8 | 18.6 | 31.7 | 120 |
| 52 | 36.8 | 5.5 | 3.7 | 6.3 | 5.5 | 11.0 | 18.9 | 32.1 | 137 |
| 55 | 44.8 | 2.4 | 4.2 | 5.6 | 4.1 | 8.1 | 14.1 | 32.4 | 60 |
| 56 | 44.6 | 1.8 | 3.7 | 5.7 | 4.1 | 7.5 | 12.9 | 39.3 | 44 |
| 57 | 47.7 | 1.2 | 3.1 | 6.8 | 5.1 | 12.1 | 20.9 | 46.5 | 31 |
| 58 | 43.9 | 1.4 | 3.4 | 6.6 | 4.8 | 11.8 | 20.6 | 152.4 | 34 |
| 59 | 46.1 | 1.1 | 3.6 | 6.5 | 4.7 | 11.6 | 20.3 | 127.6 | 28 |
| 60 | 44.6 | 1.0 | 3.8 | N/A | 4.5 | 10.1 | 17.3 | 132.5 | 25 |
| 61 | 43 | 1.1 | 4.2 | 5.8 | 4.2 | 8 | 14.3 | 49.4 | 27 |
| 62 | 46.9 | 0.6 | 4.0 | 6.7 | 5 | 8 | 14.2 | 46.3 | 14 |
| 63 | 44.6 | 0.6 | 4.2 | 6.5 | 4.6 | 10.1 | 18.4 | 50.7 | 16 |
| 64 | 43.7 | 0.7 | 4.1 | 6.3 | 4.3 | 8.1 | 14.5 | 75.4 | 19 |
| 65 | 43.1 | 0.6 | 3.6 | 5.4 | 3 | 8.6 | 15 | 46.1 | 16 |
| 66 | 46.9 | 0.6 | 2.5 | 5 | 2.7 | 7.6 | 13.6 | 39.1 | 15 |
| 67 | 45.4 | 0.7 | 3.6 | 6 | 3.5 | 7.9 | 14.3 | 69.4 | 17 |
| 68 | 44.8 | 0.8 | 3.0 | 5.7 | 3.2 | 7.4 | 12.5 | 25.5 | 20 |
| 69 | 45.4 | 0.7 | 2.0 | 6.8 | 4.8 | 11.1 | 19.1 | 39.4 | 19 |
| 70 | 44.8 | 0.6 | 3.7 | 6 | 3.8 | 7.8 | 13.7 | 31.2 | 16 |
| 71 | 42.2 | 2.1 | 4.3 | 6.4 | 4.8 | 11.1 | 20.8 | 108.4 | 53 |
| 72 | 42.3 | 0.1 | 4.4 | 6.6 | 3.2 | 7.9 | 13.8 | 39.7 | 4 |
| 73 | 41.8 | 0.1 | 4.4 | 6.6 | 3 | 7.6 | 13.1 | 25.3 | 4 |
| 74 | 41.4 | 0.9 | 4.1 | 6.6 | 5 | 9.7 | 16.5 | 34.5 | 23 |
| 75 | 42.6 | 0.2 | 3.9 | 6.7 | 3.6 | 10.2 | 17.8 | 62.3 | 4 |
| 181 | 45.4 | 0.82 | 4.3 | 6 | 4 | 9.2 | 16.1 | 34 | 21 |
| 182 | 43.1 | 0.53 | 4.3 | 6.4 | 4.9 | 9.4 | 16.5 | 54 | 13 |
| 183 | 44.6 | 0.78 | 4.1 | 6.7 | 4.7 | 9.9 | 18.2 | 53.9 | 20 |
| 184 | 43.1 | 0.89 | 3.8 | 6.8 | 4.9 | 10.1 | 18.3 | 135.4 | 22 |
| 185 | 44 | 0.96 | 3.4 | 6.5 | 4.4 | 8.9 | 15.2 | 28.7 | 24 |
| 186 | 44 | 0.9 | 3.9 | 5.9 | 3.8 | 8.4 | 14.1 | 25.7 | 23 |
| 187 | 44 | 1.3 | 3.8 | 6.0 | 4.1 | 10.5 | 17.6 | 50.8 | 34 |
| 209 | 49.2 | 6.4 | 0.2 | 6.6 | 5.9 | 7.1 | 12.7 | 23 | 159 |
| 210 | 47 | 5.6 | 0.1 | 6.6 | 6.4 | 8.6 | 15.5 | 27.7 | 141 |
| 221 | 46.1 | 0.4 | 3.3 | 6 | 3.8 | 9 | 16 | 31 | 10 |
| 222 | 46.1 | 0.5 | 3.3 | 5.5 | 3.5 | 9 | 15 | 34 | 13 |
| 223 | 46.1 | 0.5 | 3.3 | 5.2 | 3.3 | 9 | 15 | 30 | 12 |
| 225 | 48.6 | 0.8 | 0.2 | 6.6 | 4.5 | 7 | 12 | 22 | 21 |

TABLE B

| | | | | | 37° C. | | | | | | | | | | | |
|---|---|---|---|---|---|---|---|---|---|---|---|---|---|---|---|---|
| | | | | | | | | | | | | | | | (NMT | |
| | | | | | | Total | | | | | | | | | 50 μg) | |
| | 1st AQ- | | | | | DXM | Supernatant | | | d10 | d50 | d90 | | | μgs free | |
| | [DXM] | | | | Days | conc, | [DXM], | Percent | Percent Free | (diameter, | (diameter, | (diameter, | Supernatant | Internal | in a 2.5 | |
| FORMULATION # | (mg/mL) | | 1 AQ pH | LC | incubation | mg/mL | mg/ml | PPV | DXM | μm) | μm) | μm) | pH | pH | mg dose | Yield |
| 1 | 9.87 | mg/mL | 4.23 | OBLT | 0 | 2.70 | 0.19 | 42 | 4.1 | 8 | 14 | 63 | 6.5 | 5.2 | 102 | 68 |
| | | | | | 3 | 2.46 | 0.23 | 41 | 5.5 | 8 | 16 | 98 | 6.6 | 5.2 | 138 | |
| 2 | 9.77 | mg/mL | 4.82 | OBLT | 0 | 2.50 | 0.25 | 42 | 5.8 | 7 | 13 | 256 | 6.5 | 5.2 | 145 | 34 |
| | | | | | 3 | 2.42 | 0.23 | 44 | 5.3 | 7 | 11 | 76 | 6.5 | 5.2 | 133 | |
| 3 | 9.77 | mg/mL | 4.82 | OBLT | 0 | 2.30 | 0.06 | 32 | 1.8 | 13 | 235 | 478 | 6.5 | 5.1 | 44 | 60 |
| 4 | 9.77 | mg/mL | 4.82 | OBLT | 0 | 2.80 | 0.04 | 40 | 0.9 | 11 | 99 | 699 | 6.6 | 5.0 | 21 | 51 |
| 5 | 9.77 | mg/mL | 4.82 | OBLT w/1% | 0 | 3.10 | 0.04 | 37 | 0.8 | 10 | 144 | 322 | 6.6 | 5.0 | 20 | 67 |
| | | | | TO | | | | | | | | | | | | |
| 6 | 9.77 | mg/mL | 4.82 | OBLT | 0 | 3.00 | 0.04 | 36 | 0.9 | 9 | 89 | 324 | 6.6 | 5.0 | 21 | 68 |
| | | | | w/125% TC | | | | | | | | | | | | |
| 11 | 9.03 | mg/mL | 3.88 | OBLT | 0 | 2.80 | 0.10 | 44 | 2.0 | 10 | 62 | 631 | 6.5 | 5.6 | 50 | 66 |
| | | | | | 3 | 2.95 | 0.12 | 41 | 2.4 | 10 | 21 | 351 | N/A | N/A | 60 | |
| 12 | 9.03 | mg/mL | 3.88 | OBLT | 0 | 2.50 | 0.10 | 48 | 2.1 | 8 | 26 | 510 | 6.5 | 5.5 | 52 | 58 |
| | | | | | 3 | 2.40 | 0.11 | 40 | 2.8 | 8 | 15 | 168 | N/A | N/A | 69 | |
| 13 | 9.03 | mg/mL | 3.88 | OBLT | 0 | 2.20 | 0.10 | 47 | 2.4 | 7 | 14 | 208 | 6.5 | 5.4 | 60 | 62 |
| | | | | | 3 | 2.09 | 0.12 | 38 | 3.5 | 7 | 14 | 273 | N/A | N/A | 87 | |
| 14 | 9.03 | mg/mL | 3.88 | OBLT | 0 | 2.10 | 0.10 | 45 | 2.6 | 8 | 32 | 305 | 6.5 | 5.3 | 65 | 53 |
| | | | | | 3 | 2.08 | 0.15 | 37 | 4.5 | 8 | 15 | 168 | N/A | N/A | 114 | |
| 15 | 9.03 | mg/mL | 3.88 | OBLT | 0 | 2.30 | 0.10 | 48 | 2.3 | 6 | 40 | 479 | 6.5 | 5.5 | 57 | 62 |
| | | | | | 3 | 2.04 | 0.12 | 39 | 3.6 | 7 | 12 | 190 | N/A | N/A | 90 | |
| 16 | 9.03 | mg/mL | 3.88 | OBLT | 0 | 2.50 | 0.20 | 45 | 4.4 | 8 | 19 | 194 | 6.5 | N/A | 110 | 52 |
| | | | | | 3 | 1.97 | 0.15 | 38 | 4.7 | 8 | 14 | 167 | N/A | N/A | 118 | |
| 17 | 9.03 | mg/mL | 3.88 | OBLT | 0 | 2.20 | 0.10 | 48 | 2.4 | 7 | 61 | 357 | 6.5 | N/A | 59 | 62 |
| | | | | | 3 | 2.27 | 0.12 | 36 | 3.4 | 7 | 12 | 175 | N/A | N/A | 85 | |
| 18 | 9.03 | mg/mL | 3.88 | OBLT | 0 | 2.30 | 0.20 | 46 | 4.7 | 7 | 18 | 183 | 6.6 | N/A | 117 | 52 |
| | | | | | 3 | 1.99 | 0.13 | 37 | 4.1 | 8 | 18 | 232 | N/A | N/A | 103 | |
| 19 | 9.03 | mg/mL | 3.88 | OBLT | 0 | 2.16 | 0.07 | 46 | 1.8 | 6 | 12 | 166 | 6.6 | N/A | 44 | 59 |
| | | | | | 3 | 2.26 | 0.11 | 37 | 3.1 | 7 | 15 | 395 | N/A | N/A | 77 | |
| 20 | 9.03 | mg/mL | 3.88 | OBLT | 0 | 2.39 | 0.06 | 49 | 1.3 | 7 | 12 | 150 | 6.6 | N/A | 32 | 58 |
| | | | | | 3 | 2.31 | 0.11 | 39 | 2.9 | 8 | 17 | 429 | N/A | N/A | 73 | |
| 21 | 9.30 | mg/mL | 4.06 | OBLT | 0 | 2.67 | 0.11 | 42 | 2.4 | 10 | 28 | 248 | 6.5 | N/A | 60 | 66 |
| | | | | (DOPC sub) | | | | | | | | | | | | |
| 22 | 9.30 | mg/mL | 4.06 | OBLT | 0 | 2.63 | 0.07 | 47 | 1.4 | 7 | 12 | 194 | 6.5 | 5.3 | 35 | 64 |
| | | | H₃PO₄ | | 3 | 2.86 | 0.11 | 40 | 2.3 | 7 | 17 | 340 | | | 58 | |
| 23 | 9.30 | mg/mL | 4.06 | OBLT + 1% | 0 | 2.68 | 0.07 | 47 | 1.4 | 7 | 12 | 203 | 6.5 | 5.4 | 35 | 63 |
| | | | | TO | 3 | 2.70 | 0.10 | 41 | 2.2 | 8 | 21 | 309 | | | | |
| 24 | 9.30 | mg/mL | 4.06 | OBLT + 10% | 0 | 2.78 | 0.15 | 44 | 3.0 | 9 | 170 | 406 | 6.5 | 5.4 | 76 | 71 |
| | | | | TO | 3 | 3.07 | 0.15 | 39 | 3.0 | 9 | 84 | 283 | | | 75 | |
| 25 | 9.30 | mg/mL | 4.06 | EXP | 0 | 2.68 | 0.24 | 38 | 5.6 | 14 | 176 | 472 | 6.5 | 5.6 | 139 | 64 |
| | | | | | 3 | 3.19 | 0.23 | 32 | 4.9 | 13 | 94 | 477 | | | 123 | |
| 26 | 9.30 | mg/mL | 4.06 H₃PO₄ | 50/50 | 0 | 2.57 | 0.09 | 45 | 1.9 | 7 | 15 | 258 | 6.5 | 5.3 | 48 | 71 |
| | | | | EXP/OBLT | 3 | 2.80 | 0.11 | 38 | 2.4 | 8 | 38 | 251 | | | 61 | |
| 27 | n/a | | 1.80 | API in | 0 | 4.61 | 0.05 | 48 | 0.6 | 7 | 12 | 19 | 6.0 | 4.4 | 14 | 103 |
| | | | | organic | | | | | | | | | | | | |
| | | | | | 3 | 4.48 | 0.04 | 45 | 0.5 | 8 | 13 | 46 | 5.9 | | N/A | |
| 28 | n/a | | 1.80 | API in | 0 | 3.51 | 0.07 | 47 | 1.1 | 8 | 13 | 54 | 5.8 | 4.2 | 26 | 78 |
| | | | | organic | | | | | | | | | | | | |
| 29 | 9.48 | mg/mL | 4.04 | OBLT | 0 | 2.59 | 0.30 | 41 | 6.8 | 8 | 14 | 58 | 6.2 | 5.2 | 171 | 69 |
| | | | | (hypotonic | 3 | 2.63 | 0.27 | 38 | 6.3 | 8 | 16 | 103 | 6.2 | 5.2 | 159 | |
| | | | | 2^{nd} AQ) | | | | | | | | | | | | |
| 33 | 9.48 | mg/mL | 4.04 | OBLT | 0 | 2.69 | 0.23 | 41 | 5.0 | 9 | 16 | 74 | 6.3 | 5.2 | 126 | 73 |
| | | | | (hypotonic | 3 | 2.67 | 0.22 | 39 | 5.0 | 9 | 17 | 122 | 6.4 | 5.1 | 125 | |
| | | | | 2^{nd} AQ) | | | | | | | | | | | | |
| 34 | 9.48 | mg/mL | 4.04 | OBLT | 0 | 2.68 | 0.22 | 40 | 4.9 | 9 | 17 | 89 | 6.3 | 5.2 | 123 | 72 |
| | | | | (hypotonic | 3 | 2.60 | 0.23 | 37 | 5.5 | 9 | 17 | 105 | 6.3 | 5.2 | 139 | |
| | | | | 2^{nd} AQ) | | | | | | | | | | | | |
| 35 | 9.31 | mg/mL | 3.11 | OBLT 2nd | 0 | 2.82 | 0.28 | 41 | 5.9 | 7 | 14 | 96 | 6.1 | 5.0 | 147 | 78 |
| | | | | AQ | 3 | 2.86 | 0.28 | 39 | 6.0 | 8 | 16 | 140 | 6.1 | 5.0 | 150 | |
| | | | | (98 mOsm) | | | | | | | | | | | | |
| 36 | 9.44 | mg/mL | 3.59 | OBLT 2nd | 0 | 2.90 | 0.24 | 41 | 4.8 | 7 | 13 | 77 | 6.3 | 5.1 | 121 | 79 |
| | | | | AQ | 3 | 2.94 | 0.25 | 39 | 5.2 | 8 | 15 | 107 | 6.2 | 5.2 | 130 | |
| | | | | (98 mOsm) | | | | | | | | | | | | |
| 37 | 9.31 | mg/mL | 3.11 | OBLT 2nd | 0 | 2.75 | 0.07 | 44 | 1.4 | 6 | 11 | 121 | 6.9 | 5.1 | 35 | 74 |
| | | | | AQ | 3 | 2.70 | 0.09 | 39 | 2.0 | 7 | 14 | 289 | 6.7 | 5.2 | 51 | |
| | | | | (173 mOsm) | | | | | | | | | | | | |
| 38 | 9.31 | mg/mL | 3.11 | OBLT 2nd | 0 | 2.79 | 0.07 | 43 | 1.4 | 7 | 11 | 124 | 6.6 | 5.1 | 36 | 85 |
| | | | | AQ | 3 | 2.78 | 0.10 | 40 | 2.2 | 7 | 13 | 298 | 6.6 | 5.1 | 54 | |
| | | | | (126 mOsm) | | | | | | | | | | | | |
| 39 | 9.31 | mg/mL | 3.11 | OBLT 2nd | 0 | 2.76 | 0.06 | 45 | 1.2 | 6 | 12 | 244 | 6.8 | 5.2 | 30 | 85 |
| | | | | AQ | 3 | 2.79 | 0.10 | 40 | 2.2 | 7 | 12 | 189 | 6.7 | 5.2 | 54 | |
| | | | | (150 mOsm) | | | | | | | | | | | | |
| 42 | 9.92 | mg/mL | 2.07 | OBLT 2nd | 0 | 3.48 | 0.06 | 45 | 1.0 | 8 | 16 | 314 | 5.8 | 2.4 | 24 | 84 |
| | | | | AQ | 3 | 2.86 | 0.43 | 32 | 10.2 | 9 | 85 | 398 | N/A | 2.4 | N/A | |
| | | | | (150 mOsm) | | | | | | | | | | | | |
| 43 | 9.92 | mg/mL | 2.50 | OBLT 2nd | 0 | 3.16 | 0.03 | 44 | 0.5 | 7 | 13 | 74 | 6.1 | 3.4 | 13 | 87 |
| | | | | AQ | 3 | 3.24 | 0.12 | 36 | 2.4 | 8 | 16 | 166 | N/A | 3.3 | 59 | |
| | | | | (150 mOsm) | | | | | | | | | | | | |
| 44 | 9.31 | mg/mL | 3.11 H₃PO₄ | OBLT 2nd | 0 | 2.6 | 0.09 | 44.6 | 1.9 | 6 | 11 | 102 | 6.5 | 4.9 | 47 | 71 |
| | | | | AQ | 3 | 2.6 | 0.14 | 37.5 | 3.4 | 7 | 25 | 327 | N/A | N/A | 84 | |
| | | | | (150 mOsm) | 7 | 2.6 | 0.15 | 37.4 | 3.7 | 8 | 27 | 259 | | | 92 | |
| 45 | 13.45 | mg/mL | 3.08 | OBLT 2nd | 0 | 3.8 | 0.12 | 44.1 | 1.8 | 7 | 12 | 111 | 6.6 | 5.0 | 44 | 75 |
| | | | | AQ | 3 | 3.6 | 0.15 | 39.1 | 2.5 | 9 | 59 | 286 | | | 63 | |
| | | | | (150 mOsm) | 7 | 3.5 | 0.19 | 36.9 | 3.4 | 9 | 57 | 274 | | | 86 | |
| 46 | 14.91 | mg/mL | 3.30 | OBLT 2nd | 0 | 4.1 | 0.13 | 43.3 | 1.8 | 8 | 15 | 107 | 6.7 | 5.1 | 45 | 77 |
| | | | | AQ | 3 | 3.8 | 0.19 | 38.5 | 3.1 | 9 | 24 | 204 | | | 76 | |
| | | | | (150 mOsm) | 7 | 4.1 | 0.25 | 37.9 | 3.8 | 8 | 19 | 177 | | | 95 | |
| 31 | 0 | mg/mL | 1.8 H₃PO₄ | OBLT | 0 | 4.2 | 0.05 | 48.9 | 0.6 | 8 | 14 | 26 | 5.5 | 3.8 | 15 | 96 |
| | | | | 10 mg/mL | 3 | 4.1 | 0.07 | 46.4 | 0.9 | | | | | | 23 | |
| | | | | DXM | 7 | 4.1 | 0.10 | 44.3 | 1.4 | | | | | | | |
| 32 | 0 | mg/mL | 1.72 H₃PO₄ | OBLT | 4mox5C | 3.7 | 0.07 | 45.1 | 1.0 | 8 | 13 | 33 | 5.4 | 3.4 | 25 | 94 |
| | | | | 10 mg/mL | 3 | 4.1 | 0.10 | 45.5 | 1.4 | 8 | 13 | 28 | 5.4 | 3.4 | 34 | |
| | | | | DXM | 7 | 4.2 | 0.17 | 43.9 | 2.3 | 8 | 14 | 72 | 5.1 | 3.4 | 57 | |
| 32 | 0 | mg/mL | 1.72 H₃PO₄ | OBLT | 1dx5C | 3.6 | 0.01 | 47.0 | 0.2 | 8 | 14 | 86 | 6.9 | 4.7 | 5 | 94 |
| | | | | 10 mg/mL | 3 | 4.1 | 0.16 | 41.7 | 2.3 | 8 | 14 | 109 | 6.6 | 5.1 | 58 | |
| | | | | DXM | 7 | 4.4 | 0.28 | 50.8 | 3.2 | 8 | 14 | 104 | 6.5 | 5.3 | 80 | |
| 47 | 0 | mg/mL | 2.03 H₃PO₄ | OBLT + 10% | 0 | 3.7 | 0.16 | 43.5 | 2.4 | 8 | 13 | 24 | 5.6 | 4.2 | 61 | 91 |
| | | | | TO | 3 | 3.8 | 0.21 | 41.3 | 3.2 | 8 | 14 | 38 | 5.5 | 3.9 | 80 | |
| | | | | 10 mg/mL | 7 | 4.2 | 0.23 | 39.0 | 3.3 | 8 | 15 | 40 | 5.4 | 3.9 | 83 | |
| | | | | DXM | | | | | | | | | | | | |
| 48 | 0 | mg/mL | 2.03 H₃PO₄ | OBLT + 55 | 0 | 4.0 | 0.06 | 44.2 | 0.8 | 8 | 13 | 44 | 6.2 | 4.3 | 21 | |
| | | | | mol % Chol | 3 | 3.9 | 0.06 | 44.4 | 0.8 | 8 | 41 | 105 | 6.0 | 3.9 | 21 | |
| | | | | 10 mg/mL | | | | | | | | | | | | |
| | | | | DXM | 7 | 3.7 | 0.08 | 42.4 | 1.2 | 8 | 15 | 122 | 5.9 | 3.9 | 31 | |
| 49 | 0 | mg/mL | 2.03 H₃PO₄ | OBLT + 25% | 0 | 3.9 | 0.15 | 44.9 | 2.1 | 8 | 13 | 23 | 5.7 | 4.1 | 53 | |
| | | | | sub TO w/TO | 3 | 4.0 | 0.18 | 41.5 | 2.6 | 9 | 15 | 59 | 5.5 | 3.8 | 65 | |
| | | | | 10 mg/mL | 7 | 4.2 | 0.20 | 40.0 | 2.9 | 9 | 15 | 43 | 5.4 | 3.8 | 72 | |
| | | | | DXM | | | | | | | | | | | | |
| 50 | 0 | mg/mL | 2.03 H₃PO₄ | OBLT + 25% | 0 | 3.8 | 0.20 | 44.9 | 2.9 | 8 | 13 | 24 | 6.0 | 4.6 | 73 | |
| | | | | sub TO w/TO | 3 | 4.0 | 0.23 | 39.7 | 3.4 | 9 | 15 | 73 | 5.8 | 4.3 | 86 | |
| | | | | 10 mg/mL | 7 | 3.9 | 0.28 | 37.5 | 4.5 | 9 | 17 | 126 | 5.6 | 4.3 | 113 | |
| | | | | DXM | | | | | | | | | | | | |
| 55 | 0 | mg/mL | 2.03 H₃PO₄ | OBLT | 0 | 4.2 | 0.18 | 44.8 | 2.4 | 8 | 14 | 32 | 5.6 | 4.1 | 59 | |
| | | | | 10 mg/mL | 3 | 3.6 | 0.14 | 41.5 | 2.3 | 9 | 17 | 138 | 5.5 | 3.8 | 57 | |
| | | | | DXM | 7 | 3.9 | 0.14 | 40.6 | 2.1 | 9 | 16 | 89 | 5.5 | 3.8 | 52 | |
| 56 | 0 | mg/mL | 2.03 H₃PO₄ | OBLT + 60 mol | 0 | 3.7 | 0.12 | 44.6 | 1.8 | 8 | 13 | 39 | 5.7 | 4.1 | 45 | |
| | | | | % Chol | 3 | 3.7 | 0.07 | 42.8 | 1.1 | 8 | 15 | 126 | 5.8 | 3.8 | 28 | |
| | | | | 10 mg/mL | 7 | 3.8 | 0.09 | 42.4 | 1.3 | 8 | 15 | 163 | 5.7 | 3.8 | 33 | |
| | | | | DXM | | | | | | | | | | | | |
| 57 | 0 | mg/mL | 1.81 H₃PO₄ | OBLT | 0 | 3.32 | 0.08 | 44.8 | 1.3 | 12 | 21 | 47 | 6.8 | 5.1 | 33 | 75.8 |
| | | | 50 mM | 10 mg/mL | 3 | 3.26 | 0.13 | 41.5 | 2.3 | 13 | 23 | 93 | 6.6 | 5.0 | 58 | |
| | | | | DXM | 7 | 3.24 | 0.17 | 40.6 | 3.1 | 14 | 23 | 67 | 6.5 | 5.1 | 78 | |
| 58 | 0 | mg/mL | 1.82 H₃PO₄ | OBLT | 0 | 3.36 | 0.08 | 44.8 | 1.3 | 12 | 21 | 152 | 6.6 | 4.8 | 33 | 66.5 |
| | | | 55 mM | 10 mg/mL | 3 | 3.36 | 0.10 | 41.5 | 1.7 | 12 | 23 | 230 | 6.6 | 4.8 | 44 | |
| | | | | DXM | 7 | 3.97 | 0.13 | 40.6 | 1.9 | 12 | 21 | 124 | 6.4 | 4.8 | 48 | |
| 59 | 0 | mg/mL | 1.81 H₃PO₄ | OBLT | 0 | 3.66 | 0.07 | 44.8 | 1.1 | 12 | 20 | 128 | 6.5 | 4.7 | 28 | 75.4 |
| | | | | 10 mg/mL | 3 | 3.78 | 0.08 | 41.5 | 1.3 | 11 | 19 | 122 | 6.6 | 4.8 | 33 | |
| | | | 60 mM | DXM | 7 | 3.75 | 0.11 | 40.6 | 1.7 | 12 | 21 | 176 | 6.4 | 4.7 | 43 | |
| 60 | 0 | mg/mL | 1.81 H₃PO₄ | OBLT | 0 | 3.77 | 0.07 | 44.8 | 1.0 | 10 | 17 | 133 | — | 4.5 | 25 | 80 |
| | | | 65 mM | 10 mg/mL | 3 | 3.84 | 0.06 | 41.5 | 0.9 | 10 | 18 | 195 | 6.5 | 4.6 | 24 | |
| | | | | DXM | 7 | 3.76 | 0.08 | 40.6 | 1.2 | 10 | 17 | 107 | 6.4 | 4.5 | 31 | |
| 61 | 0 | mg/mL | H₃PO₄ | OBLT | 0 | 4.00 | 0.08 | 43.0 | 1.1 | 8 | 14 | 49 | 5.8 | 4.2 | 29 | 81 |
| | | | 70 mM | 10 mg/mL | 3 | 3.92 | 0.06 | 43.1 | 0.9 | 8 | 15 | 114 | 6.1 | 4.2 | 22 | |
| | | | | DXM | | | | | | | | | | | | |
| 62 | 0 | mg/mL | H₃PO₄ | OBLT | 0 | 4.16 | 0.05 | 46.9 | 0.6 | 8 | 14 | 46 | 6.7 | 5.0 | 16 | 88 |
| | | | 70 mM | 10 mg/mL | 3 | 4.33 | 0.06 | 44.6 | 0.8 | 8 | 14 | 40 | 6.7 | 5.0 | 19 | |
| | | | | DXM | | | | | | | | | | | | |
| 63 | 0 | mg/mL | H₃PO₄ | OBLT 150% | 0 | 4.18 | 0.05 | 44.6 | 0.7 | 10 | 18 | 51 | 6.5 | 4.6 | 17 | 82 |
| | | | 70 mM | 10 mg/mL | 3 | 4.05 | 0.04 | 43.7 | 0.6 | 10 | 19 | 52 | 6.6 | 4.6 | 14 | |
| | | | | DXM | | | | | | | | | | | | |
| 64 | 0 | mg/mL | HH₃PO₄ | OBLT 125% | 0 | 4 | 0.05 | 43.7 | 0.7 | 8 | 15 | 75 | 6.3 | 4.3 | 18 | 86 |
| | | | 70 mM | 10 mg/mL | 3 | 4.04 | 0.04 | 46.1 | 0.5 | 8 | 15 | 60 | 6.4 | 4.3 | 13 | |
| | | | | DXM | | | | | | | | | | | | |
| 65 | 0 | mg/mL | H₃PO₄ | OBLT 125% | 0 | 3.47 | 0.04 | 43.1 | 0.7 | 9 | 15 | 46 | 5.4 | 3.0 | 16 | 83 |
| | | | 70 mM | (5.5/4.5; 1 | 3 | 3.46 | 0.03 | 44.6 | 0.5 | 9 | 16 | 36 | 5.6 | 3.1 | 12 | |
| | | | | AQ/DCM) SA | | | | | | | | | | | | |
| | | | | Addition | | | | | | | | | | | | |
| | | | | 10 mg/mL | | | | | | | | | | | | |
| | | | | DXM | | | | | | | | | | | | |
| 66 | 0 | mg/mL | H₃PO₄ | OBLT 150% | 0 | 2.64 | 0.03 | 46.9 | 0.6 | 8 | 14 | 39 | 5.0 | 2.7 | 15 | 85 |
| | | | 70 mM | (6/4; 1 | 3 | 2.61 | 0.04 | 45.4 | 0.8 | 8 | 15 | 47 | 5.0 | 2.8 | 21 | |
| | | | | AQ/DCM) SA | | | | | | | | | | | | |
| | | | | Addition | | | | | | | | | | | | |
| | | | | 10 mg/mL | | | | | | | | | | | | |
| | | | | DXM | | | | | | | | | | | | |
| 67 | 0 | mg/mL | H₃PO₄ | OBLT 125% | 0 | 3.63 | 0.05 | 45.4 | 0.7 | 8 | 14 | 69 | 6.0 | 3.5 | 17 | 90 |
| | | | 70 mM | (5.5/4.5; 1 | 3 | 3.53 | 0.03 | 43.3 | 0.4 | 8 | 14 | 99 | 5.9 | 3.5 | 11 | |
| | | | | AQ/DCM) | | | | | | | | | | | | |
| | | | | Slow | | | | | | | | | | | | |
| | | | | Addition | | | | | | | | | | | | |
| | | | | 10 mg/mL | | | | | | | | | | | | |
| | | | | DXM | | | | | | | | | | | | |
| 68 | 0 | mg/mL | H₃PO₄ | OBLT 150% | 0 | 2.99 | 0.04 | 44.8 | 0.8 | 7 | 13 | 26 | 5.7 | 3.2 | 20 | 89 |
| | | | 70 mM | (6/4; 1 | 3 | 2.94 | 0.03 | 43.3 | 0.5 | 7 | 12 | 22 | 5.6 | 3.1 | 13 | |
| | | | | AQ/DCM) | | | | | | | | | | | | |
| | | | | Slow | | | | | | | | | | | | |
| | | | | Addition | | | | | | | | | | | | |
| | | | | 10 mg/mL | | | | | | | | | | | | |
| | | | | DXM | | | | | | | | | | | | |
| 69 | 0 | mg/mL | H₃PO₄ | OBLT Low | 0 | 2.01 | 0.03 | 45.4 | 0.7 | 11 | 19 | 39 | 6.8 | 4.8 | 19 | 77 |
| | | | 35 mM | Potency, 5 | 3 | 2.00 | 0.04 | 44.8 | 1.0 | 11 | 19 | 38 | 6.5 | 4.7 | 25 | |
| | | | | mg/ml 1st AQ | | | | | | | | | | | | |
| | | | | 5 mg/mL | | | | | | | | | | | | |
| | | | | DXM | | | | | | | | | | | | |
| 70 | 0 | mg/mL | H₃PO₄ | OBLT(150%)/ | 0 | 3.66 | 0.04 | 44.8 | 0.6 | 8 | 14 | 31 | 6.0 | 3.8 | 16 | 89 |
| | | | 70 mM | EXP (150%); | 3 | 3.74 | 0.02 | 44.1 | 0.3 | 8 | 14 | 41 | 6.1 | 3.8 | 8 | |
| | | | | (5.5/4.5; 1 | | | | | | | | | | | | |
| | | | | AQ/DCM) | | | | | | | | | | | | |
| | | | | Slow | | | | | | | | | | | | |
| | | | | Addition | | | | | | | | | | | | |
| | | | | 10 mg/mL | | | | | | | | | | | | |
| | | | | DXM | | | | | | | | | | | | |
| 71 | 0 | mg/mL | H₃PO₄ | DSPC/DEPC | 0 | 4.01 | 0.15 | 42.2 | 2.1 | 11 | 21 | 108 | 6.4 | 4.8 | 53 | 71 |
| | | | 70 mM | 25/75 | 3 | 4.06 | 0.13 | 42.8 | 1.8 | 11 | 22 | 137 | 6.4 | 4.8 | 45 | |
| | | | | (4.5/5.5; | | | | | | | | | | | | |
| | | | | 1AQ/DCM) | | | | | | | | | | | | |
| | | | | 10 mg/mL | | | | | | | | | | | | |
| | | | | DXM | | | | | | | | | | | | |
| 72 | 0 | mg/mL | H₃PO₄ | OBLT | 0 | 4.11 | 0.01 | 42.3 | 0.1 | 8 | 14 | 40 | 6.6 | 3.2 | 4 | 92 |
| | | | 87.5 mM | 10 mg/mL | 3 | 4.00 | 0.01 | 42.0 | 0.1 | 8 | 13 | 33 | 6.5 | 3.5 | 4 | |
| | | | | DXM | | | | | | | | | | | | |
| 73 | 0 | mg/mL | H₃PO₄ | OBLT | 0 | 4.11 | 0.01 | 41.8 | 0.1 | 8 | 13 | 25 | 6.6 | 3.0 | 4 | 85 |
| | | | 100 mM | 10 mg/mL | 3 | 4.32 | 0.01 | 42.0 | 0.1 | 8 | 13 | 26 | 6.2 | 3.2 | 3 | |
| | | | | DXM | | | | | | | | | | | | |
| 74 | 0 | mg/mL | H₃PO₄ | EXP 150% | 0 | 3.79 | 0.06 | 41.4 | 0.9 | 10 | 17 | 35 | 6.6 | 5.0 | 23 | 85 |
| | | | 70 mM | 10 mg/mL | 3 | 3.89 | N/A | 40.0 | | 9 | 16 | 33 | 6.7 | 4.9 | | |
| | | | | DXM | | | | | | | | | | | | |
| 75 | 0 | mg/mL | H₃PO₄ | EXPAREL | 0 | 3.71 | 0.01 | 42.6 | 0.2 | 10 | 18 | 62 | 6.7 | 3.6 | 4 | 89 |
| | | | 70 mM | 150% + 7 | 3 | 3.84 | N/A | 40.6 | | 9 | 17 | 63 | 6.6 | 3.9 | | |
| | | | | mg/ml TO | | | | | | | | | | | | |
| | | | | (13.5 mg/ml/2 | | | | | | | | | | | | |
| | | | | 8.6 mM total) | | | | | | | | | | | | |
| | | | | 10 mg/mL | | | | | | | | | | | | |
| | | | | DXM | | | | | | | | | | | | |
| 181 | 10 | mg/mL | H₃PO₄ | 125% OBLT, | 0 | 4.34 | 0.07 | 45.4 | 0.82 | 9 | 16 | 34 | 6.2 | 3.9 | 21 | 89.9 |
| | | | 80 mM | 1.6:1 | 3 | 4.10 | 0.03 | 44.1 | 0.44 | 9 | 16 | 35 | 6.3 | 3.9 | 11 | |
| | | | | | 7 | 4.20 | 0.05 | 42.6 | 0.74 | 9 | 18 | 38 | 6.1 | 3.87 | 19 | |
| 182 | 10 | mg/mL | H₃PO₄ | 125% OBLT, | 0 | 4.1 | 0.04 | 43.1 | 0.53 | 9 | 17 | 54 | 6.5 | 4.3 | 13 | 89 |
| | | | 75 mM | 1.5:1 | 3 | 4.1 | 0.03 | 42.4 | 0.43 | 10 | 17 | 39 | 6.5 | 4.2 | 11 | |
| | | | | | 7 | 4.1 | 0.05 | 42.6 | 0.71 | 10 | 18 | 147 | 6.4 | 4.2 | 18 | |
| 183 | 10 | mg/mL | H₃PO₄ | 125% OBLT, | 0 | 4.1 | 0.06 | 44.6 | 0.78 | 10 | 18 | 54 | 6.7 | 4.7 | 20 | 75.5 |
| | | | 70 mM | 1.4:1 | 3 | 4.6 | 0.06 | 43.1 | 0.75 | 10 | 18 | 62 | 6.7 | 4.7 | 19 | |
| | | | | | 7 | 4.1 | 0.07 | 42.6 | 0.94 | 10 | 19 | 68 | 6.6 | 4.7 | 23 | |
| 184 | 10 | mg/mL | H₃PO₄ | 125% OBLT, | 0 | 3.6 | 0.06 | 43.1 | 0.89 | 10 | 18 | 135 | 6.8 | 4.8 | 22 | 74.1 |
| | | | 65 mM | 1.3:1 | 3 | 3.7 | 0.06 | 41.5 | 0.97 | 10 | 18 | 91 | 6.8 | 4.8 | 24 | |
| | | | | | 7 | 3.8 | 0.08 | 42.4 | 1.19 | 11 | 20 | 300 | 6.6 | 4.8 | 30 | |
| 185 | 10 | mg/mL | H₃PO₄ | 125% OBLT, | 0 | 3.3 | 0.06 | 44.1 | 0.96 | 9 | 15 | 29 | 6.5 | 4.4 | 24 | 69 |
| | | | 75 mM | 1.5:1 | 3 | 3.6 | 0.03 | 42.03 | 0.55 | 9 | 15 | 30 | 6.5 | 4.2 | 14 | |
| | | | | | 7 | 3.9 | 0.07 | 41.98 | 1.13 | 9 | 15 | 33 | 6.1 | 4.4 | 28 | |
| 186 | 10 | mg/mL | H₃PO₄ | 100% OBLT, | 0 | 3.8 | 0.06 | 44.1 | 0.92 | 8 | 14 | 26 | 5.9 | 3.8 | 23 | 89 |
| | | | 75 mM | 1.5:1 | 3 | 3.8 | 0.03 | 43.08 | 0.51 | 9 | 15 | 29 | 6.0 | 3.7 | 13 | |
| | | | | | 7 | 3.8 | 0.06 | 42.42 | 0.97 | 9 | 15 | 40 | 5.7 | 3.7 | 24 | |
| 187 | 10 | mg/mL | H₃PO₄ | 75% OBLT, | 0 | 3.7 | 0.09 | 44.1 | 1.34 | 11 | 18 | 51 | 6.0 | 4.1 | 34 | 80 |
| | | | 75 mM | 1.5:1 | 3 | 3.9 | 0.06 | 42.42 | 0.92 | 10 | 17 | 48 | 6.0 | 4.0 | 23 | |
| | | | | | 7 | 4.1 | 0.09 | 40.63 | 1.27 | 10 | 18 | 100 | 5.8 | 4.0 | 32 | |
| 209 | 1 | mg/mL | H₃PO₄ | 125% OBLT, | 0 | 0.25 | 0.03 | 49.2 | 6.38 | 7 | 13 | 23 | 6.6 | 5.9 | 159 | 49 |
| | | | 7.5 mM | low pot. | 3 | 0.24 | 0.03 | 48.4 | 5.99 | 7 | 14 | 24 | 6.7 | 6.0 | 150 | |
| | | | | 1 mg · ml | 7 | 0.26 | 0.06 | 62.9 | 8.23 | 9 | 16 | 30 | 6.4 | 6.0 | 206 | |
| 210 | 0.5 | mg/mL | H₃PO₄ | 125% OBLT, | 0 | 0.13 | 0.01 | 47 | 5.64 | 9 | 16 | 28 | 6.6 | 6.4 | 141 | 65 |
| | | | 3.75 mM | low pot. | 3 | 0.13 | 0.02 | 53.8 | 6.42 | 9 | 17 | 31 | 6.8 | 6.4 | 161 | |
| | | | | 0.5 mg · ml | 7 | 0.12 | 0.04 | 77.3 | 6.57 | 12 | 22 | 41 | 6.2 | 6.0 | 164 | |
| 221 | 10 | mg/mL | H₃PO₄ | 125% OBLT | 0 | 3.43 | 0.026 | 46.1 | 0.41 | 9 | 16 | 31 | 6.0 | 3.8 | 10 | 80 |
| | | | 65 mM | 4 mg/ml | 3 | 3.36 | 0.02 | 46.1 | 0.37 | 9 | 16 | 34 | 5.9 | 3.8 | 9 | |
| | | | | DPPG | 7 | 3.58 | 0.04 | 44.6 | 0.62 | 9 | 16 | 29 | 5.4 | 3.5 | | |
| 222 | 10 | mg/mL | H₃PO₄ | 125% OBLT | 0 | 3.35 | 0.033 | 46.1 | 0.53 | 9 | 15 | 34 | 5.5 | 3.5 | 13 | 81 |
| | | | 70 mM | 4 mg/ml | 3 | 3.29 | 0.03 | 46.8 | 0.45 | 9 | 15 | 28 | 5.6 | 3.5 | 11 | |
| | | | | DPPG | 7 | 3.37 | 0.11 | 45.6 | 1.78 | 9 | 15 | 30 | 5.1 | 3.3 | 44 | |
| 223 | 10 | mg/mL | H₃PO₄ | 125% OBLT | 0 | 3.35 | 0.031 | 46.1 | 0.50 | 9 | 15 | 30 | 5.2 | 3.3 | 12 | 81 |
| | | | 75 mM | 4 mg/ml | 3 | 3.49 | 0.03 | 46 | 0.45 | 9 | 16 | 29 | 5.3 | 3.2 | 11 | |
| | | | | DPPG | 7 | 3.79 | 0.06 | 43.7 | 0.89 | 9 | 16 | 77 | 4.8 | 3.1 | 22 | |
| 225 | 0.5 | mg/mL | H₃PO₄ | 125% OBLT | 0 | 0.25 | 0.004 | 48.6 | 0.82 | 7 | 12 | 22 | 6.6 | 4.5 | 21 | 76 |
| | | | 12.5 mM | 0.5 mg/ml | 3 | 0.25 | 0.00 | 45.7 | 0.66 | 7 | 13 | 24 | 6.6 | 4.5 | 17 | |

While the present application has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A multivesicular liposome formulation comprising:
dexmedetomidine encapsulated in a first aqueous component of the multivesicular liposomes;
a lipid component comprising at least one amphipathic lipid and at least one neutral lipid; and
one or more pH modifying agents.

2. The multivesicular liposome formulation of claim 1, further comprising less than 2% by weight of unencapsulated dexmedetomidine.

3. The multivesicular liposome formulation of claim or 2, wherein the multivesicular liposomes further comprise one or more tonicity agents.

4. The multivesicular liposome formulation of any one of claims 1 to 3, wherein the multivesicular liposomes further comprise cholesterol and/or a plant sterol.

5. The multivesicular liposome formulation of any one of claims 1 to 4, wherein the amphipathic lipid comprises a phosphatidylcholine or a salt thereof, a phosphatidylglycerol or a salt thereof, or combinations thereof.

6. The multivesicular liposome formulation of claim 5, wherein the phosphatidylglycerol is DPPG.

7. The multivesicular liposome formulation of claim 5, wherein the phosphatidylcholine is selected from the group consisting of DEPC, DSPC, DMPC, DOPC, or combinations thereof.

8. The multivesicular liposome formulation of any one of claims 1 to 7, wherein the neutral lipid comprises triglyceride, propylene glycol ester, ethylene glycol ester, or squalene, or combinations thereof.

9. The multivesicular liposome formulation of claim 8, wherein the neutral lipid comprises triglyceride.

10. The multivesicular liposome formulation of claim 8 or 9, wherein the triglyceride comprises triolein or tricaprylin, or a combination thereof.

11. The multivesicular liposome formulation of any one of claims 1 to 10, wherein the pH modifying agents comprise organic acids, organic bases, inorganic acids, or inorganic bases, or combinations thereof.

12. The multivesicular liposome formulation of claim 11, wherein at least one pH modifying agent resides in the first aqueous component of the multivesicular liposomes and said pH modifying agent comprises an inorganic acid.

13. The multivesicular liposome formulation of claim 12, wherein the inorganic acid comprises phosphoric acid.

14. The multivesicular liposome formulation any on of claims 1 to 13, wherein the formulation is a liquid suspension comprising multivesicular liposomes suspended in a liquid suspending medium.

15. The multivesicular liposome formulation of claim 14, wherein the liquid suspending medium is a buffered saline solution.

16. The multivesicular liposome formulation of claim 14 or 15, wherein the concentration of dexmedetomidine in the particle suspension is from about 0.1 mg/mL to about 20 mg/mL.

17. The multivesicular liposome formulation claim 16, wherein the concentration of dexmedetomidine in the particle suspension is from about 2.5 mg/mL to about 8 mg/mL.

18. The multivesicular liposome formulation of claim 17, wherein the concentration of dexmedetomidine in the liquid suspension is from about 3.0 mg/mL to about 5 mg/mL.

19. The multivesicular liposome formulation of any one of claims 1 to 18, wherein the multivesicular liposomes have an internal pH from about 2.0 to about 8.0.

20. The multivesicular liposome formulation of claim 19, wherein the multivesicular liposomes have an internal pH from about 2.5 to about 6.5.

21. The multivesicular liposome formulation of claim 20, wherein the multivesicular liposomes have an internal pH from about 3.0 to about 5.5.

22. The multivesicular liposome formulation of any one of claims 1 to 21, wherein the multivesicular liposomes have an external pH range from about 3.0 to about 7.5.

23. The multivesicular liposome formulation of claim 22, wherein the multivesicular liposomes have an external pH range from about 4.0 to about 7.0.

24. The multivesicular liposome formulation of any one of claims 1 to 23, wherein the multivesicular liposomes are stable at 37° C. for at least 3 days.

25. A pharmaceutical composition comprising the multivesicular liposomes formulation of any one of claims 1 to 24, wherein the composition comprises equal to or less about 500 micrograms of unencapsulated dexmedetomidine.

26. The pharmaceutical composition of claim 25, wherein the composition is for administration in a single injection.

27. The pharmaceutical composition of claim 25 or 26, wherein the composition provides sustained release of dexmedetomidine for 2 to 12 days.

28. The pharmaceutical composition of claim 27, wherein the composition provides sustained release of dexmedetomidine for 3 to 7 days.

29. A method for treating or ameliorating pain or anxiety comprising administering a pharmaceutical composition of any one of claims 25 to 28 to a subject in need thereof.

30. A method for inducing arousable sedation comprising administering a pharmaceutical composition of any one of claims 25 to 28 to a subject in need thereof.

31. The method of claim 29 or 30, wherein the administration is parenteral.

32. The method of claim 31, wherein the parenteral administration is selected from the group consisting of subcutaneous injection, tissue injection, intramuscular injection, spinal injection, intraocular injection, epidural injection, intrathecal injection, intraotic injection, and perineural injection, and combinations thereof.

33. The method of claim 32, wherein the parenteral administration is subcutaneous injection or tissue injection.

34. A process for preparing dexmedetomidine encapsulated multivesicular liposomes, the process comprising:
mixing a first aqueous component with a lipid component comprising at least one organic solvent, at least one amphipathic lipid, and at least one neutral lipid to form a first water-in-oil emulsion, wherein at least one of the first aqueous component and the lipid component comprises dexmedetomidine;
combining the first water-in-oil emulsion with a second aqueous component to form a second emulsion; and
substantially removing the organic solvent from the second emulsion to form multivesicular liposomes.

35. The process of claim 34, further comprising diluting the second emulsion in a third aqueous solution prior to substantially removing the organic solvent.

36. The process of claim 34 or 35, wherein the organic solvent is substantially removed by exposing the second emulsion in a gas atmosphere.

37. The process of any one of claims 34 to 36, further comprising isolating the multivesicular liposome particles and suspending them in a liquid suspending medium to form a suspension of multivesicular liposomes.

38. The process of claim 37, wherein the liquid suspending medium is a buffered saline solution.

39. The process of any one of claims 34 to 38, wherein the first aqueous component comprises dexmedetomidine and at least one pH modifying agent.

40. The process of claim 39, wherein the volume of the lipid component is greater than the volume of the first aqueous component.

41. The process of any one of claims 34 to 38, wherein the lipid component comprises dexmedetomidine.

42. The process of claim 41, wherein the volume of the lipid component and the volume of the first aqueous component is about 1:1.

43. The process of any one of claims 34 to 42, wherein the pH range of the first aqueous component is from about 1.0 to about 6.0.

44. The process of claim 43, wherein the pH range of the first aqueous component is from about 2.0 to about 5.0.

45. The process of claim 44, wherein the osmolality of the first aqueous component is from about 280 mOsm/kg to 310 mOsm/kg.

46. The process of any one of claims 34 to 45, wherein the second aqueous component comprises at least one pH modifying agent and at least one tonicity agent.

47. The process of claim 46, wherein the tonicity agent comprises sorbitol, sucrose, or dextrose, or combinations thereof.

48. The process of claim 46 or 47, wherein osmolality of the second aqueous component is from about 80 mOsm/kg to about 500 mOsm/kg.

49. The process of claim 48, wherein the osmolality of the second aqueous component is from about 150 mOsm/kg to about 190 mOsm/kg.

50. The process of any one of claims 34 to 49, wherein the pH range of the second aqueous component is from about 6.0 to about 11.5.

51. The process of claim 50, wherein the pH range of the second aqueous component is from about 7.0 to about 11.

52. The process of any one of claims 37 to 51, wherein the concentration of dexmedetomidine in the suspension is from about 0.1 mg/mL to about 20 mg/mL.

53. The process claim 52, wherein the concentration of dexmedetomidine in the suspension is from about 2.5 mg/mL to about 8 mg/mL.

54. The process of claim 53, wherein the concentration of dexmedetomidine in the suspension is from about 3 mg/mL to about 5 mg/mL.

55. The process of any one of claims 52 to 54, wherein unencapsulated DXM is about 2% or less by weight of total amount of dexmedetomidine in the suspension.

56. A pharmaceutical composition comprising dexmedetomidine encapsulated multivesicular liposomes prepared by the process of claims 34 to 55.
